(19) United States
(12) Patent Application Publication
(10) Pub. No.: US 2023/0346661 A1
(43) Pub. Date: Nov. 2, 2023

(54) Anhydrous Azelaic Acid Topical Formulations

(71) Applicant: Baek Clinical Inc., Los Angeles, CA (US)

(72) Inventor: Jihoon P. Baek, Los Angeles, CA (US)

(21) Appl. No.: 17/918,826

(22) PCT Filed: Apr. 16, 2021
(86) PCT No.: PCT/US2021/027835

§ 371 Date: Oct. 13, 2022

Related U.S. Application Data
(60) Provisional application No. 63/010,889, filed on Apr. 16, 2020.

(51) Int. Cl. A61K 8/362; A61K 8/42; A61Q 19/00

(52) U.S. Cl. CPC A61K 8/362; A61K 8/42; A61Q 19/00; A61K 2800/31; A61K 2800/87

(57) ABSTRACT

Topical formulations of azelaic acid dissolved in a combination of a urea agent and a non-aqueous skin-compatible solvent are provided. The formulations are completely solubilized in the composition and have desirable physical properties, such as non-grittiness. The topical formulations can include high concentrations of azelaic acid. Topical compositions of this disclosure find use in treating or preventing a variety of cosmetic and/or dermatological conditions as well as to reduce the appearance of skin discolorations.

This application claims the benefit of U.S. Provisional Application No. 63/010,889, filed Apr. 16, 2020, the entire disclosure of which is hereby incorporated by reference in its entirety.

### FIELD OF INVENTION

Compositions and methods for treating, preventing, or improving dermatocosmetic conditions, including reducing the appearance of skin discolorations.

### INTRODUCTION

Azelaic acid (also commonly known as nonanedioic acid) is a dicarboxylic acid that exists naturally in many whole grains and is widely used in topical compositions to treat or prevent a range of cosmetic and/or dermatological conditions (listed below) as well as reduce the appearance of dyschromia/uneven pigmentation. Non-limiting examples of such conditions of dermatocosmetic conditions that may be improved by topical application of the compositions of the present invention include: melasma, inflammatory dermatoses (including acne, rosacea, melasma, psoriasis).

Tyrosinase is a copper-containing enzyme that catalyzes the production of melanin and other pigments from tyrosine by oxidation. Azelaic acid has several mechanisms of depigmenting the skin, including tyrosinase inhibition. Its selective action is also believed to be due to its specific cytotoxic and antiproliferative effects towards abnormal melanocytes, via inhibiting DNA synthesis and mitochondrial enzymes. Q H Nguyen et al, Int J Dermatol. Vol. 34, pp 75-84 (1995). The “gold standard” in cosmetic dermatology for skin lightening/brightening is hydroquinone. However, hydroquinone (HQ) is known to have side effects including allergic sensitization and exogenous ochronosis (blue-black pigmentation of the skin caused by long-term use of hydroquinone). Azelaic acid is also used to lighten the appearance of the skin—including for example, melasma—but with a more favorable safety profile (i.e., fewer side effects). See, e.g., LM Balina et al, Int J Dermatol. Vol 30, pp 893-5 (1991) (65% good or excellent results in lightening melasma from use of 20% azelaic acid with no significant treatment differences were observed with regard to overall rating, reduction in lesion size, and pigmentary intensity when compared to 4% hydroquinone; no allergic sensitization or exogenous ochronosis observed with azelaic acid).

The scientific and patent literature describe azelaic acid as penetrating the skin more easily and causing less irritation when completely solubilized. See, e.g., U.S. Pat. No. 5,925,679. Dispersions deliver azelaic acid in an undissolved state. When applied to the skin, undissolved azelaic acid is not readily absorbed and as a result an excess of azelaic acid must be present to be effective. The higher the concentration of azelaic acid, the more likely irritation (burning, stinging and redness) to the skin will occur.

Furthermore, scientific and patent literature describe azelaic acid as being difficult to solubilize. See, e.g., U.S. Pat. No. 5,925,679. While azelaic acid is somewhat soluble in water, cosmetic oils and alcohols, each of these solvents has serious limitations. Water only marginally dissolves azelaic acid so that a water and azelaic acid solution would contain a maximum of about 0.24% by weight (w/w) azelaic acid, not likely enough to be effective. Azelaic acid has little or no solubility in cosmetic oils. Alcohols are good solvents but are unsatisfactory because large amounts of alcohol e.g., isopropyl alcohol, in a topical composition has the undesirable side effect of drying the skin, and reducing the stability of azelaic acid in the composition. Indeed, some alcohols e.g., ethyl alcohol, render azelaic acid unstable at normal temperatures resulting in a totally ineffective composition. South Korean Patent KR100861978B1 confirms the challenges of solubilizing clinically effective amounts of azelaic acid.

There has been and remains a need for an azelaic acid delivery vehicle that is fully solubilized, non-gritty, and non-oily/non-greasy. These needs are met by the anhydrous azelaic acid formulations of the present disclosure.

### SUMMARY

Topical formulations of azelaic acid dissolved in a combination of a urea agent and a non-aqueous skin-compatible solvent are provided. The topical formulations can include high concentrations of the azelaic acid of 1% to 20% by weight. Topical compositions of this disclosure find use in treating or preventing a variety of cosmetic and/or dermatological conditions.

The inventor of the present disclosure discovered that particular amounts of azelaic acid and urea composition can be added to a non-aqueous solvent to provide solubilized azelaic acid and urea at various desired concentration levels. Solubilized azelaic acid is much less likely to irritate the skin because azelaic acid in a dissolved state is much more readily absorbed by the skin than in the undissolved states found in dispersions. Better absorption means less azelaic acid need be present in the formulation to be effective thereby lowering the risk of irritation to the skin.

The inventor of the present disclosure discovered that azelaic acid in low concentrations, and concentrations higher than 2-5% can be added to a non-aqueous solvent to provide solubilized azelaic acid that eliminates burning, itching, and/or numbing sensations upon topical application.

### DETAILED DESCRIPTION

This disclosure provides topical formulations of azelaic acid dissolved in a combination of a urea agent and a non-aqueous skin-compatible solvent. The formulations are storage stable for an extended period of time without undesirable discoloration or significant degradation of the azelaic acid in the composition. This disclosure provides particular topical formulations which have been developed and optimized to provide skin compatibility and desirable physical properties.

Topical compositions of this disclosure find use in treating or preventing a variety of cosmetic and/or dermatological conditions as well as to reduce the appearance of chronological and/or environmentally-caused skin aging, such as dyschromia or uneven pigmentation, and dark circles under the eyes. Non-limiting examples of dermatocosmetic conditions that may be improved by topical application of the compositions of the present disclosure include: keratoses, melasma, lentigines, liver spots, inflammatory dermatoses (including eczema, acne, psoriasis), and xeroses (also known in the art as dry skin or pruritus).

In some embodiments, formulations of the present disclosure include the ingredients: (i) 1% to 20% by weight azelaic acid; and (ii) 1-20% urea agent; dissolved in (iii) a non-aqueous skin-compatible solvent.

### Azelaic Acid

This disclosure provides formulations that include combination of particular amount of a urea agent in a non-aqueous skin-compatible solvent which together can provide for dissolution of particular amounts of azelaic acid and which produce skin-compatible liquid compositions in which the azelaic acid is substantially stable to decomposition. In some embodiments, the amounts of azelaic acid stably dissolved in the composition are greater than would otherwise be possible without the particular combinations of ingredients provided by the disclosure.

The terms “azelaic acid” and “nonanedioic acid” refer to the naturally occurring dicarboxylic acid of CAS Registry Number: 123-99-9. Any convenient form of azelaic acid can be utilized in the subject formulations. In some embodiments, the azelaic acid used in the formulations of the present disclosure is a powder.

In certain embodiments, the azelaic acid material used in preparing the subject compositions is composed of granular particles. Such a particulate powder has a particle size (e.g., mean particle size) of less than about 25 microns, such as less than about 20 microns, and more preferably less than about 12.5 microns, e.g., as measured by a Hagman gauge. In some embodiments, all of the azelaic acid powder used in preparing the subject compositions is capable of passage through a No. 100 U.S. Standard Sieve, a standard testing procedure used by the US Pharmacopoeia. In some embodiments, 80% or more (such as 90% or more, or 100%) of azelaic acid powder used in preparing the subject composition is capable of passage through a No. 325 U.S. Standard Sieve.

In some embodiments, the amount of azelaic acid in the subject composition is at least 1% by weight, at least 2% by weight, at least 4% by weight, or about 5% by weight, such as at least about 10% by weight, at least about 12% by weight, at least about 15% by weight, at least about 20% by weight, or at least about 25% by weight. In some embodiments, the subject composition includes about 28% by weight or less of azelaic acid in the non-aqueous solvent solution, such as about 25% by weight or less. In certain embodiments, the non-aqueous solvent is 1,3-propanediol. In particular embodiments, the amount of azelaic acid in the subject composition is between about 1% by weight and about 12% by weight, or between about 4% by weight and 8% by weight, or between about 8% by weight and about 12% by weight. In some embodiments, the amount of azelaic acid in the subject composition is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight.

In particular embodiments, the amount of azelaic acid in the subject composition is between about 1% by weight and about 20% by weight (e.g., about 1%, about 5%, about 10%, about 15%, or about 20%) where the ratio of azelaic acid to urea agent (% wt ratio) is 0.8 to 9, such as a ratio of 2 (i.e., 2:1). In some embodiments, the ratio of azelaic acid to urea agent (% wt ratio) is 0.5 to 9.5, such as 0.5 to 4.5, 0.8 to 1.2, 1.2 to 1.6, 1.4 to 2.0, 2.0 to 2.4, 2.4 to 2.8, 2.8 to 3.2, 3.2 to 3.6, 3.6 to 4.0, 4.0 to 4.4, 4.4 to 4.8, 4.8 to 5.2, 5.2 to 5.6, 5.6 to 6.0, 6.0 to 6.4, 6.4 to 6.8, 6.8 to 7.2, 7.2 to 7.6, 7.6 to 8.0, 8.0 to 8.4, 8.4 to 8.8, 8.8 to 9.2, or 9.2 to 9.5.

In particular embodiments, the amount of azelaic acid in the subject composition is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, where the ratio (% wt ratio) of azelaic acid to urea agent (% wt ratio) is 1.0 (i.e., 1:1) or more, 1.25 or more, 1.50 or more, 1.75 or more, 2.0 or more, 2.5 or more, 3.0 or more, 3.25 or more, 3.50 or more, 3.75 or more, 4.0 or more, 4.25 or more, 4.50 or more, 4.75 or more, 5 or more, 6 or more, 7 or more, 8 or more, or 9 or more.

In general, the amounts of azelaic acid in a composition are calculated relative to the solution phase based on the non-aqueous solvent. See Formulations of Table 2. However, the amounts of azelaic acid and other ingredients relative to the emulsion composition as a whole can readily be calculated by the skilled artisan. Formulation 3 of Table 2 shows exemplary emulsion compositions where the % by weight values shown are relative to the total emulsion composition. It is understood that, in some cases, these concentrate solutions having particular amounts of azelaic acid can be combined with an immiscible ingredient (e.g., an oil component) and an emulsifying agent to produce an emulsion composition (e.g., as described below).

In some embodiments, the amount of azelaic acid in the subject composition is at least about 1% by weight, about 2% by weight, about 3% by weight, or about 4% by weight, such as at least about 6% by weight, at least about 8% by weight, at least about 10% by weight, at least about 12% by weight. In certain embodiments, the non-aqueous solvent is 1,3-propanediol.

It is understood that, in some cases, these concentrate solutions having particular amounts of azelaic acid can be combined with an immiscible ingredient (e.g., an oil component) and an emulsifying agent to produce an emulsion composition (e.g., as described below).

### Urea Agent

The formulations of the present disclosure include a urea agent in an amount sufficient to enhance the solubility of azelaic acid in the non-aqueous skin compatible solvent and to provide a stable solution. The inventor discovered that particular amounts of urea agent can be added to a non-aqueous solvent to provide stable solutions of azelaic acid at various desired concentration levels. These amounts of urea agent are selected based on observations regarding the maximum amount of azelaic acid that can be stably dissolved in the particular non-aqueous solvent, and minimum amounts of urea agent that should be included to provide a stable azelaic acid solution.

Urea agents of interest include, but are not limited to, urea and substituted urea, such as alkyl substituted urea, more particularly mono-substituted or di-substituted alkyl urea (e.g., hydroxyalkyl urea). In some embodiments, the urea agent is a hydroxyalkyl urea, such as hydroxyethyl urea. The urea agent ingredient used in the subject formulations can be a combination of urea and/or substituted ureas. For example, the urea agent can be a combination of urea and hydroxyethyl urea. In certain embodiments, the urea agent is urea. In certain embodiments, the urea agent is hydroxyethyl urea.

In certain embodiments, the urea agent used in preparing the subject compositions is in a crystalline form before solubilized in a solvent. In some embodiments, the crystalline form of urea has a particle size (e.g., mean particle size) of 100 microns or more, 125 microns or more, 150 microns or more, 175 microns or more, 200 microns or more, 225 microns or more, 250 microns or more, 275 microns or more, or 300 microns or more. In certain embodiments, the urea agent used in preparing the subject compositions is in a crystalline form before solubilized in a solvent. In some embodiments, the crystalline form of urea has a particle size (e.g., mean particle size) of 0.5 mm or more, 1 mm or more, 1.5 mm or more, 2.0 mm or more, 2.5 mm or more, 3 mm or more, 3.5 mm or more, 4 mm or more, 4.5 mm or more, 5 mm or more, 5.5 mm or more, 6 mm or more, 6.5 mm or more, or 7 mm or more.

In some embodiments, the percent by weight amount of urea in the composition of the present disclosure is an amount that is sufficient to solubilize azelaic acid in the non-aqueous solvent.

In some embodiments, the topical composition includes: a) 1% to 20% by weight azelaic acid; and b) 1% to 20% by weight of a urea agent, wherein the ratio (% wt ratio) of azelaic acid to urea agent is between about 0.5 and about 9.5, such as 0.5 to 4.5, 0.8 to 1.2, 1.2 to 1.6, 1.4 to 2.0, 2.0 to 2.4, 2.4 to 2.8, 2.8 to 3.2, 3.2 to 3.6, 3.6 to 4.0, 4.0 to 4.4, 4.4 to 4.8, or 4.8 to 5.2, 5.2 to 5.6, 5.6 to 6.0, 6.0 to 6.4, 6.4 to 6.8, 6.8 to 7.2, 7.2 to 7.6, 7.6 to 8.0, 8.0 to 8.4, 8.4 to 8.8, 8.8 to 9.2, or 9.2 to 9.5, dissolved in a non-aqueous skin-compatible solvent selected from polyol, C(2-6) alkanediol, glycol ether, dimethyl ether, or a combination thereof.

In some embodiments, the amount of a urea agent in the subject composition is at least about 1% by weight, at least about 2% by weight, at least about 3% by weight, at least about 4% by weight, at least about 5% by weight, such as at least about 6% by weight, at least about 7% by weight, at least about 8% by weight, at least about 9% by weight, at least about 10% by weight, at least about 11% by weight, at least about 12% by weight, at least about 14% by weight, at least about 15% by weight, at least about 16% by weight, at least about 17% by weight, at least about 18% by weight, at least about 19% by weight, or at least about 20% by weight.

In certain embodiments, the non-aqueous solvent is 1,3-propanediol. In particular embodiments, the amount of a urea agent in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of a urea agent in the subject composition is about 5%, about 10%, about 15%, about 20%, or about 25% by weight.

In particular embodiments, the amount of a urea agent in the subject composition is between about 10% by weight and about 20% by weight (e.g., about 10%, about 15%, or about 20%) where the ratio of azelaic acid to a urea agent is (% wt ratio) is 0.8 to 9.5, such as a ratio of 2 (i.e., 2:1).

In particular embodiments, the amount of urea agent in the subject composition is between about 1% by weight and about 20% by weight (e.g., about 1%, about 2%, about 3%, about 4%, about 5%, about 10%, about 15%, about 20%) where the ratio of azelaic acid to a urea agent (% wt ratio) is 0.8 to 9.5, such as a ratio of 1.25 (i.e., 1.25:1) or a ratio of 1.0 (i.e., 1:1).

In some embodiments, the subject composition includes about 5 to 20% by weight of a urea agent (e.g., about 5%, about 6%, about 7%, about 8%, about 9%, about 10%, about 11%, about 12%, about 13%, about 14%, about 15%, about 16%, about 17%, about 18%, about 19%, about 20%) and a non-aqueous solvent. In some embodiments, the subject composition includes between 1-5%, 5-10%, 10-15%, or 15-20% by weight of a urea agent, and a non-aqueous solvent. In certain embodiments, the subject composition includes about 5% by weight of a urea agent and a non-aqueous solvent. In certain embodiments, the subject composition includes about 15% by weight of a urea agent and a non-aqueous solvent. In certain embodiments, the subject composition includes about 20% by weight of a urea agent and a non-aqueous solvent.

In some embodiments, the subject composition includes about 5 to 7% by weight of a urea agent and a non-aqueous solvent. In some embodiments, the subject composition includes about 7 to 9% by weight of a urea agent and a non-aqueous solvent. In certain embodiments, the subject composition includes about 9 to 11% by weight of a urea agent and a non-aqueous solvent. In certain embodiments, the subject composition includes about 11 to 13% by weight of a urea agent and a non-aqueous solvent. In certain embodiments, the subject composition includes about 13 to 15% by weight of a urea agent and a non-aqueous solvent. In certain embodiments, the subject composition includes about 15 to 17% by weight of a urea agent and a non-aqueous solvent. In certain embodiments, the subject composition includes about 17 to 19 by weight of a urea agent and a non-aqueous solvent. In certain embodiments, the subject composition includes about 19 to 21% by weight of a urea agent and a non-aqueous solvent. In certain embodiments, the subject composition includes about 21 to 23% by weight of a urea agent and a non-aqueous solvent. In certain embodiments, the subject composition includes about 23 to 25% by weight of a urea agent and a non-aqueous solvent.

In some embodiments, the composition comprises a first phase comprising a urea agent dissolved in the non-aqueous solvent. In some embodiments, the composition comprises a first phase comprising a urea agent and azelaic acid dissolved in the non-aqueous solvent.

### Skin Compatible Solvent

In addition to the azelaic acid and urea agent (e.g., as described herein), the azelaic acid formulations of the present disclosure contain, as an essential ingredient, at least one non-aqueous skin-compatible solvent. A skin compatible solvent is a solvent that does not cause irritation or sensitization when applied topically to the skin. Non-aqueous skin-compatible solvents of interest include polyols, C₍₂₋₆₎ alkanediols, glycol ethers, dimethyl ethers, and combinations thereof.

In some embodiments, the formulation of the present disclosure comprises azelaic acid and urea agent, dissolved in a non-aqueous solvent to form a homogenous solution. In some embodiments, the formulation of the present disclosure comprises azelaic acid dissolved in a first phase solution comprising urea agent dissolved in a non-aqueous solvent. In some embodiments, the formulation of the present disclosure comprises a homogenous solution comprising azelaic acid, a urea agent, and a non-aqueous skin compatible solvent.

In some embodiments, the solvent is a skin compatible polyol. A polyol is an organic alcohol solvent having two or more hydroxy groups. In some embodiments, the polyol solvent is a C(₃₋₁₂) polyol, such as a C(₃₋₆)polyol. In some embodiments, the polyol solvent is a polyether polyol. In some embodiments, the polyol solvent is a polyester polyol. Skin compatible polyols of interest include, but are not limited to, glycerol (1,2,3-propanetriol); diglycerol; propylene glycol (1,2-propanediol); dipropylene glycol; 1,3-propanediol; butylene glycol (1,3-butanediol); 1,2-butanediol; pentylene glycol (1,2-pentanediol); 1,5-pentanediol; 1,2-hexanediol; 1,6-hexanediol; 1,2,3-hexanetriol, 1,2,6-hexanetriol; ethoxydiglycol; and dimethyl isosorbide. In some embodiments, the solvent is a glycol ether, a dimethyl ether, or a combination thereof. A preferred skin-compatible solvent is 1,3-propanediol, commercially available from DuPont Tate & Lyle BioProducts LLC under the tradename ZEMEA®. Other preferred skin-compatible solvents include butylene glycol and, to a lesser degree, propylene glycol.

In some embodiments, the subject composition includes about 10 to 99% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more) of a non-aqueous skin compatible solvent. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol and one or more additional skin compatible solvents.

In some embodiments, the solvent is a skin compatible polyol. A polyol is an organic alcohol solvent having two or more hydroxy groups. In some embodiments, the polyol solvent is a C(₃₋₁₂)polyol, such as a C(₃₋₆)polyol. In some embodiments, the polyol solvent is a C₍₂₋₆₎ alkanediol. In some embodiments, the polyol solvent is a polyether polyol. In some embodiments, the polyol solvent is a polyester polyol. Skin compatible polyols of interest include, but are not limited to, glycerol (1,2,3-propanetriol); diglycerol; propylene glycol (1,2-propanediol); dipropylene glycol; 1,3-propanediol; butylene glycol (1,3-butanediol); 1,2-butanediol; pentylene glycol (1,2-pentanediol); 1,5-pentanediol; 1,2-hexanediol; 1,6-hexanediol; 1,2,3-hexanetriol, 1,2,6-hexanetriol; ethoxydiglycol; and dimethyl isosorbide. In some embodiments, the solvent is a glycol ether, a dimethyl ether, or a combination thereof. A preferred skin-compatible solvent is 1,3-propanediol, commercially available from DuPont Tate & Lyle BioProducts LLC under the tradename ZEMEA®. In some embodiments, the solvent is a mixture of 1,3 propanediol and 1,2 hexanediol. In some embodiments, the subject composition includes about 1 to 20% by weight of a urea agent, about 1 to 20% by weight of azelaic acid, and 1,3-propanediol.

In some embodiments, the subject composition includes about 10 to 80% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, or about 80% or more) of a non-aqueous skin compatible solvent. In some embodiments, the subject composition includes about 1 to 25% by weight of a azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 25% of a urea agent (about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%); and 10 to 80% polyol. In some embodiments, the subject composition includes between 5-10%, about 10-15%, about 15-20%, about 20-25%, or about 25-30% by weight of a azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); between about 1-5%, about 5-10%, about 10-15%, or about 15-20% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and a polyol. In certain embodiments, the subject composition includes about 5% by weight of a urea agent and 10% to 80% by weight of polyol. In certain embodiments, the subject composition includes about 15% by weight of a azelaic acid; 15% by weight of a urea agent; and 10% to 80% by weight of polyol. In certain embodiments, the subject composition includes about 20% by weight of a urea agent; about 20% by weight of azelaic acid; and 10% to 80% by weight of polyol.

In some embodiments, the subject composition includes about 7 to 9% by weight azelaic acid, 7 to 9% by weight of a urea agent, and 10% to 80% by weight of polyol. In certain embodiments, the subject composition includes about 9 to 11% by weight azelaic acid, 7 to 9% by weight of a urea agent, and 10% to 80% by weight of polyol. In certain embodiments, the subject composition includes about 11 to 13% by weight azelaic acid, about 11 to 13% by weight urea agent, and 10% to 80% by weight of polyol. In certain embodiments, the subject composition includes about 11 to 13% by weight azelaic acid, about 13 to 15% by weight urea agent, and 10% to 80% by weight of polyol. In certain embodiments, the subject composition includes about 15 to 17% by weight azelaic acid, about 15 to 17% by weight urea agent and 10% to 80% by weight of polyol. In certain embodiments, the subject composition includes about 17 to 19 by weight azelaic acid, about 17 to 19 by weight urea agent, and 10% to 80% by weight of polyol. In certain embodiments, the subject composition includes about 19 to 21% by weight azelaic acid, about 19 to 21% by weight urea agent, and 10% to 80% by weight of polyol. In certain embodiments, the subject composition includes about 21 to 23% by weight azelaic acid, about 21 to 23% by weight urea agent and 10% to 80% by weight of polyol. In certain embodiments, the subject composition includes about 23 to 25% by weight azelaic acid, about 23 to 25% by weight urea agent, and 10% to 80% by weight of polyol.

In some embodiments, the subject composition includes about 5 to 30% by weight of azelaic acid (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 5 to 30% by weight of a urea agent (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and a non-aqueous solvent selected from the group consisting of: C(₂₋₆) alkanediols, glycol ethers, dimethyl ethers. In some embodiments, the subject composition includes between about 5-10%, about 10-15%, or about 15-20% by weight of azelaic acid; about 5-10%, about 10-15%, or about 15-20% by weight of a urea agent; and a non-aqueous solvent selected from the group consisting of: C(₂₋₆) alkanediols, glycol ethers, dimethyl ethers. In certain embodiments, the subject composition includes about 5% by weight of azelaic acid, 5% by weight of a urea agent, and a non-aqueous solvent selected from the group consisting of: C(₂₋₆) alkanediols, glycol ethers, dimethyl ethers. In certain embodiments, the subject composition includes about 15% by weight of azelaic acid, about 15% by weight of a urea agent, and a polyol. In certain embodiments, the subject composition includes about 20% by weight of azelaic acid, about 20% by weight of a urea agent, and a non-aqueous solvent selected from the group consisting of: C(2-6) alkanediols, glycol ethers, dimethyl ethers.

In some embodiments, the subject composition includes about 5 to 7% by weight of azelaic acid, 5 to 7% by weight of urea agent, and 1,3-propanediol. In some embodiments, the subject composition includes about 5 to 7% by weight of azelaic acid, about 7 to 9% by weight urea agent, and a non-aqueous solvent selected from the group consisting of: C(₂₋₆) alkanediols, glycol ethers, dimethyl ethers. In certain embodiments, the subject composition includes about 9 to 11% by weight urea agent and a non-aqueous solvent selected from the group consisting of: C(₂₋₆) alkanediols, glycol ethers, dimethyl ethers. In certain embodiments, the subject composition includes about 11 to 13% by weight azelaic acid, about 11 to 13% by weight urea agent, and a non-aqueous solvent selected from the group consisting of: C(₂₋₆) alkanediols, glycol ethers, dimethyl ethers. In certain embodiments, the subject composition includes about 13 to 15% by weight azelaic acid, about 13 to 15% by weight urea agent and a non-aqueous solvent selected from the group consisting of: C(₂₋₆) alkanediols, glycol ethers, dimethyl ethers. In certain embodiments, the subject composition includes about 15 to 17% by weight azelaic acid, about 15 to 17% by weight urea agent, and a non-aqueous solvent selected from the group consisting of: C(₂₋₆) alkanediols, glycol ethers, dimethyl ethers. In certain embodiments, the subject composition includes about 17 to 19% by weight azelaic acid, about 17 to 19 by weight urea agent and a non-aqueous solvent selected from the group consisting of: C(₂₋₆) alkanediols, glycol ethers, dimethyl ethers. In certain embodiments, the subject composition includes about 19 to 21% by weight azelaic acid, about 19 to 21% by weight urea agent and a non-aqueous solvent selected from the group consisting of: C(₂₋₆) alkanediols, glycol ethers, dimethyl ethers. In certain embodiments, the subject composition includes about 21 to 23% by weight azelaic acid, about 21 to 23% by weight urea agent and 10% to 80% by weight of polyol. In certain embodiments, the subject composition includes about 23 to 25% by weight azelaic acid, about 23 to 25% by weight urea agent and a non-aqueous solvent selected from the group consisting of: C(₂₋₆) alkanediols, glycol ethers, dimethyl ethers.

In some embodiments, the subject composition includes about 1 to 30% by weight azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%), and 10% to 80% by weight of polyether polyol. In some embodiments, the subject composition includes between 1-5%, 5-10%, about 10-15%, or about 15-20% by weight of azelaic acid; 1-5%, 5-10%, 10-15%, or 15-20% by weight of a urea agent; and 10% to 80% by weight of polyether polyol. In certain embodiments, the subject composition includes about 5% by weight of azelaic acid; about 5% by weight of a urea agent; and 10% to 80% by weight of polyether polyol. In certain embodiments, the subject composition includes about 15% by weight of azelaic acid; about 15% by weight of a urea agent; and 10% to 80% by weight of polyether polyol. In certain embodiments, the subject composition includes about 20% by weight azelaic acid; about 20% by weight of a urea agent; and 10% to 80% by weight of polyether polyol.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of polyester polyol. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of polyester polyol. In certain embodiments, the subject composition includes about 5% by weight of azelaic acid; about 5% by weight of a urea agent and 10% to 80% by weight of polyester polyol. In certain embodiments, the subject composition includes about 15% by weight of azelaic acid; about 15% by weight of a urea agent; and 10% to 80% by weight of polyester polyol. In certain embodiments, the subject composition includes about 20% by weight of azelaic acid; about 20% by weight of a urea agent; and 10% to 80% by weight of polyester polyol.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of glycerol (1,2,3-propanetriol). In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of glycerol (1,2,3-propanetriol). In certain embodiments, the subject composition includes about 5% by weight of azelaic acid; about 5% by weight of a urea agent and 10% to 80% by weight of glycerol (1,2,3-propanetriol). In certain embodiments, the subject composition includes about 15% by weight of azelaic acid; about 15% by weight of a urea agent; and 10% to 80% by weight of glycerol (1,2,3-propanetriol). In certain embodiments, the subject composition includes about 20% by weight of azelaic acid; about 20% by weight of a urea agent; and 10% to 80% by weight of glycerol (1,2,3-propanetriol).

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of diglycerol. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of diglycerol. In certain embodiments, the subject composition includes about 5% by weight of azelaic acid; about 5% by weight of a urea agent and 10% to 80% by weight of diglycerol. In certain embodiments, the subject composition includes about 15% by weight of azelaic acid; about 15% by weight of a urea agent; and 10% to 80% by weight of diglycerol 1. In certain embodiments, the subject composition includes about 20% by weight of azelaic acid; about 20% by weight of a urea agent; and 10% to 80% by weight of diglycerol.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of propylene glycol (1,2-propanediol). In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of propylene glycol (1,2-propanediol). In certain embodiments, the subject composition includes about 5% by weight of azelaic acid; about 5% by weight of a urea agent and 10% to 80% by weight of propylene glycol (1,2-propanediol). In certain embodiments, the subject composition includes about 15% by weight of azelaic acid; about 15% by weight of a urea agent; and 10% to 80% by weight of propylene glycol (1,2-propanediol). In certain embodiments, the subject composition includes about 20% by weight of azelaic acid; about 20% by weight of a urea agent; and 10% to 80% by weight of propylene glycol (1,2-propanediol).

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of dipropylene glycol. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of dipropylene glycol. In certain embodiments, the subject composition includes about 5% by weight of azelaic acid; about 5% by weight of a urea agent and 10% to 80% by weight of dipropylene glycol. In certain embodiments, the subject composition includes about 15% by weight of azelaic acid; about 15% by weight of a urea agent; and 10% to 80% by weight of dipropylene glycol. In certain embodiments, the subject composition includes about 20% by weight of azelaic acid; about 20% by weight of a urea agent; and 10% to 80% by weight of dipropylene glycol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) azelaic acid; about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of 1,3-propanediol. In some embodiments, the subject composition includes between about 1-5%, 5-10%, about 10-15%, or about 15-20% by weight of azelaic acid; 1-5%, 5-10%, 10-15%, or 15-20% by weight of a urea agent, and 10% to 80% by weight of 1,3-propanediol. In certain embodiments, the subject composition includes about 1% by weight of azelaic acid; about 5% by weight of a urea agent and 10% to 80% by weight of 1,3-propanediol. In certain embodiments, the subject composition includes about 2% by weight of azelaic acid; about 5% by weight of a urea agent and 10% to 80% by weight of 1,3-propanediol. In certain embodiments, the subject composition includes about 3% by weight of azelaic acid; about 5% by weight of a urea agent and 10% to 80% by weight of 1,3-propanediol. In certain embodiments, the subject composition includes about 4% by weight of azelaic acid; about 5% by weight of a urea agent and 10% to 80% by weight of 1,3-propanediol. In certain embodiments, the subject composition includes about 5% by weight of azelaic acid; about 5% by weight of a urea agent and 10% to 80% by weight of 1,3-propanediol. In certain embodiments, the subject composition includes about 15% by weight of azelaic acid; about 15% by weight of a urea agent and 10% to 80% by weight of 1,3-propanediol. In certain embodiments, the subject composition includes about 20% by weight of azelaic acid; about 20% by weight of a urea agent and 10% to 80% by weight of 1,3-propanediol. In some embodiments, the subject composition includes about 5 to 7% by weight of azelaic acid; about 5 to 7% by weight urea agent and 1,3-propanediol. In some embodiments, the subject composition includes about 7 to 9% by weight of azelaic acid; about 7 to 9% by weight urea agent and 1,3-propanediol. In certain embodiments, the subject composition includes about 9 to 11% by weight of azelaic acid; about 9 to 11% by weight urea agent and 1,3-propanediol. In certain embodiments, the subject composition includes about 11 to 13% by weight of azelaic acid; about 11 to 13% by weight urea agent and 1,3-propanediol. In certain embodiments, the subject composition includes about 13 to 15% by weight of azelaic acid; about 13 to 15% by weight urea agent and 1,3-propanediol. In certain embodiments, the subject composition includes about 15 to 17% by weight of azelaic acid; about 15 to 17% by weight urea agent and 1,3-propanediol. In certain embodiments, the subject composition includes about 17 to 19% by weight of azelaic acid; about 17 to 19% by weight urea agent and 1,3-propanediol. In certain embodiments, the subject composition includes about 19 to 21% by weight of azelaic acid; about 19 to 21% by weight urea agent and 1,3-propanediol. In certain embodiments, the subject composition includes about 21 to 23% by weight of azelaic acid; about 21 to 23% by weight urea agent and 1,3-propanediol. In certain embodiments, the subject composition includes about 23 to 25% by weight of azelaic acid; about 23 to 25% by weight urea agent and 1,3-propanediol.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of butylene glycol (1,3-butanediol). In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of butylene glycol (1,3-butanediol). In certain embodiments, the subject composition includes about 5% by weight of azelaic acid; about 5% by weight of a urea agent and 10% to 80% by weight of butylene glycol (1,3-butanediol). In certain embodiments, the subject composition includes about 15% by weight of azelaic acid; about 15% by weight of a urea agent; and 10% to 80% by weight of butylene glycol (1,3-butanediol). In certain embodiments, the subject composition includes about 20% by weight of azelaic acid; about 20% by weight of a urea agent; and 10% to 80% by weight of butylene glycol (1,3-butanediol).

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of 1,2-butanediol; pentylene glycol (1,2-pentanediol). In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of 1,2-butanediol; pentylene glycol (1,2-pentanediol). In certain embodiments, the subject composition includes about 5% by weight of azelaic acid; about 5% by weight of a urea agent and 10% to 80% by weight of 1,2-butanediol; pentylene glycol (1,2-pentanediol). In certain embodiments, the subject composition includes about 15% by weight of azelaic acid; about 15% by weight of a urea agent; and 10% to 80% by weight of 1,2-butanediol; pentylene glycol (1,2-pentanediol). In certain embodiments, the subject composition includes about 20% by weight of azelaic acid; about 20% by weight of a urea agent; and 10% to 80% by weight of 1,2-butanediol; pentylene glycol (1,2-pentanediol).

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of 1,5-pentanediol. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of 1,5-pentanediol. In certain embodiments, the subject composition includes about 5% by weight of azelaic acid; about 5% by weight of a urea agent and 10% to 80% by weight of 1,5-pentanediol. In certain embodiments, the subject composition includes about 15% by weight of azelaic acid; about 15% by weight of a urea agent; and 10% to 80% by weight of 1,5-pentanediol. In certain embodiments, the subject composition includes about 20% by weight of azelaic acid; about 20% by weight of a urea agent; and 10% to 80% by weight of 1,5-pentanediol.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of 1,2-hexanediol. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of 1,2-hexanediol. In certain embodiments, the subject composition includes about 5% by weight of azelaic acid; about 5% by weight of a urea agent and 10% to 80% by weight of polyester polyol. In certain embodiments, the subject composition includes about 15% by weight of azelaic acid; about 15% by weight of a urea agent; and 10% to 80% by weight of 1,2-hexanediol. In certain embodiments, the subject composition includes about 20% by weight of azelaic acid; about 20% by weight of a urea agent; and 10% to 80% by weight of 1,2-hexanediol.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of 1,6-hexanediol. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of 1,6-hexanediol. In certain embodiments, the subject composition includes about 5% by weight of azelaic acid; about 5% by weight of a urea agent and 10% to 80% by weight of 1,6-hexanediol. In certain embodiments, the subject composition includes about 15% by weight of azelaic acid; about 15% by weight of a urea agent; and 10% to 80% by weight of 1,6-hexanediol. In certain embodiments, the subject composition includes about 20% by weight of azelaic acid; about 20% by weight of a urea agent; and 10% to 80% by weight of 1,6-hexanediol.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of 1,2,3-hexanetriol. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of 1,2,3-hexanetriol. In certain embodiments, the subject composition includes about 5% by weight of azelaic acid; about 5% by weight of a urea agent and 10% to 80% by weight of 1,2,3-hexanetriol. In certain embodiments, the subject composition includes about 15% by weight of azelaic acid; about 15% by weight of a urea agent; and 10% to 80% by weight of 1,2,3-hexanetriol. In certain embodiments, the subject composition includes about 20% by weight of azelaic acid; about 20% by weight of a urea agent; and 10% to 80% by weight of 1,2,3-hexanetriol.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of 1,2,6-hexanetriol. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of 1,2,6-hexanetriol. In certain embodiments, the subject composition includes about 5% by weight of azelaic acid; about 5% by weight of a urea agent and 10% to 80% by weight of 1,2,6-hexanetriol. In certain embodiments, the subject composition includes about 15% by weight of azelaic acid; about 15% by weight of a urea agent; and 10% to 80% by weight of 1,2,6-hexanetriol. In certain embodiments, the subject composition includes about 20% by weight of azelaic acid; about 20% by weight of a urea agent; and 10% to 80% by weight of 1,2,6-hexanetriol.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of ethoxydiglycol. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of ethoxydiglycol. In certain embodiments, the subject composition includes about 5% by weight of azelaic acid; about 5% by weight of a urea agent and 10% to 80% by weight of ethoxydiglycol. In certain embodiments, the subject composition includes about 15% by weight of azelaic acid; about 15% by weight of a urea agent; and 10% to 80% by weight of ethoxydiglycol. In certain embodiments, the subject composition includes about 20% by weight of azelaic acid; about 20% by weight of a urea agent; and 10% to 80% by weight of ethoxydiglycol.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of dimethyl isosorbide. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of dimethyl isosorbide. In certain embodiments, the subject composition includes about 5% by weight of azelaic acid; about 5% by weight of a urea agent and 10% to 80% by weight of dimethyl isosorbide. In certain embodiments, the subject composition includes about 15% by weight of azelaic acid; about 15% by weight of a urea agent; and 10% to 80% by weight of dimethyl isosorbide. In certain embodiments, the subject composition includes about 20% by weight of azelaic acid; about 20% by weight of a urea agent; and 10% to 80% by weight of dimethyl isosorbide.

In some embodiments, the subject composition includes about 1 to 30% by weight of azelaic acid (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%); and 10% to 80% by weight of polyester polyol. In some embodiments, the subject composition includes 1-5%, 5-10%, 10-15%, or 15-20% by weight of azelaic acid; 1-5%, 5-10%, 10-15%, or 15-20% by weight of a urea agent; and 10% to 80% by weight of glycol ether, dimethyl ether, or a combination thereof. In certain embodiments, the subject composition includes about 5% by weight of azelaic acid; about 5% by weight of a urea agent and 10% to 80% by weight of glycol ether, dimethyl ether, or a combination thereof. In certain embodiments, the subject composition includes about 15% by weight of azelaic acid; about 15% by weight of a urea agent; and 10% to 80% by weight of polyester polyol. In certain embodiments, the subject composition includes about 20% by weight of azelaic acid; about 20% by weight of a urea agent; and 10% to 80% by weight of glycol ether, dimethyl ether, or a combination thereof.

In some embodiments, the ratio of azelaic acid to urea in the liquid composition is 1.5 to 3.0. In some embodiments, the ratio of azelaic acid to urea in the liquid composition is 2.0 to 3.0. In some embodiments, the ratio of azelaic acid to urea in the liquid composition is 2.0 to 2.5. In some embodiments, the ratio of azelaic acid to urea in the liquid composition is 2.0 to 2.5, where the weight percent of azelaic acid is 8% or more, such as 10% or more (e.g., 10% to 15%, or 10% to 12% azelaic acid), dissolved in a solvent component that is composed of one or more C₍₂₋₆₎ alkanediols. In some embodiments, the solvent component is composed of 1,3-propanediol, or a mixture of 1,3-propanediol and 1,2-hexanediol. In some embodiments, the ratio of azelaic acid to urea in the liquid composition is 2.0.

In some embodiments, the formulation of the present disclosure comprises a first phase that is a homogenous solution comprising a urea agent and a non-aqueous skin compatible solvent. In certain embodiments, the composition comprises a second phase solution that comprises azelaic acid and the first phase solution. In some embodiments, the formulations of the present disclosure comprise a first phase comprising about 1% or more, about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 16% or more, about 17% or more, about 18% or more, about 19% or more, about 20% or more, about 21% or more, about 22% or more, about 23% or more, about 24% or more, about 25% or more, about 26% or more, about 27% or more, about 28% or more, about 29% or more, about 30% or more, about 31% or more, about 32% or more, about 33% or more, about 34% or more, about 35% or more, about 36% or more, about 37% or more, about 38% or more, about 39% or more, about 40% or more, about 41% or more, about 42% or more, about 43% or more, about 44% or more, about 45% or more, about 46% or more, about 47% or more, about 48% or more, about 49% or more, or about 50% or more by weight of the urea agent; and about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, or about 80% or more by weight of a non-aqueous skin-compatible solvent.

In some embodiments, the first phase includes about 10 to 80% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, or about 80% or more) of a non-aqueous skin compatible solvent. In some embodiments, the first phase includes about 5 to 30% by weight of a urea agent (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 80% polyol. In some embodiments, the first phase includes 1-5%, 5-10%, 10-15%, or 15-20% by weight of a urea agent, and a polyol. In certain embodiments, the first phase includes about 5% by weight of a urea agent and 10% to 80% by weight of polyol. In certain embodiments, the first phase includes about 15% by weight of a urea agent and 10% to 80% by weight of polyol. In certain embodiments, the first phase includes about 20% by weight of a urea agent and 10% to 80% by weight of polyol.

In some embodiments, the first phase includes about 7 to 9% by weight urea agent and 10% to 80% by weight of polyol. In certain embodiments, the first phase includes about 9 to 11% by weight urea agent and 10% to 80% by weight of polyol. In certain embodiments, the first phase includes about 11 to 13% by weight urea agent and 10% to 80% by weight of polyol. In certain embodiments, the first phase includes about 13 to 15% by weight urea agent and 10% to 80% by weight of polyol. In certain embodiments, the first phase includes about 15 to 17% by weight urea agent and 10% to 80% by weight of polyol. In certain embodiments, the first phase includes about 17 to 19% by weight urea agent and 10% to 80% by weight of polyol. In certain embodiments, the first phase includes about 19 to 21% by weight urea agent and 10% to 80% by weight of polyol. In certain embodiments, the first phase includes about 21 to 23% by weight urea agent and 10% to 80% by weight of polyol. In certain embodiments, the first phase includes about 23 to 25% by weight urea agent and 10% to 80% by weight of polyol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and a non-aqueous solvent selected from the group consisting of: C(₂₋₆) alkanediols, glycol ethers, dimethyl ethers. In some embodiments, the first phase includes 1-5%, 5-10%, 10-15%, or 15-20% by weight of a urea agent, and a non-aqueous solvent selected from the group consisting of: C(₂₋₆) alkanediols, glycol ethers, dimethyl ethers. In certain embodiments, the first phase includes about 5% by weight of a urea agent and a non-aqueous solvent selected from the group consisting of: C(₂₋₆) alkanediols, glycol ethers, dimethyl ethers. In certain embodiments, the first phase includes about 15% by weight of a urea agent and a polyol. In certain embodiments, the first phase includes about 20% by weight of a urea agent and a non-aqueous solvent selected from the group consisting of: C(₂₋₆) alkanediols, glycol ethers, dimethyl ethers.

In some embodiments, the first phase includes about 5 to 7% by weight of a urea agent and 1,3-propanediol. In some embodiments, the first phase includes about 7 to 9% by weight urea agent and a non-aqueous solvent selected from the group consisting of: C(2-6) alkanediols, glycol ethers, dimethyl ethers. In certain embodiments, the first phase includes about 9 to 11% by weight urea agent and a non-aqueous solvent selected from the group consisting of: C(₂₋₆) alkanediols, glycol ethers, dimethyl ethers. In certain embodiments, the first phase includes about 11 to 13% by weight urea agent and a non-aqueous solvent selected from the group consisting of: C(₂₋₆) alkanediols, glycol ethers, dimethyl ethers. In certain embodiments, the first phase includes about 13 to 15% by weight urea agent and a non-aqueous solvent selected from the group consisting of: C(₂₋₆) alkanediols, glycol ethers, dimethyl ethers. In certain embodiments, the first phase includes about 15 to 17% by weight urea agent and a non-aqueous solvent selected from the group consisting of: C(₂₋₆) alkanediols, glycol ethers, dimethyl ethers. In certain embodiments, the first phase includes about 17 to 19% by weight urea agent and a non-aqueous solvent selected from the group consisting of: C(₂₋₆) alkanediols, glycol ethers, dimethyl ethers. In certain embodiments, the first phase includes about 19 to 21% by weight urea agent and a non-aqueous solvent selected from the group consisting of: C(₂₋₆) alkanediols, glycol ethers, dimethyl ethers. In certain embodiments, the first phase includes about 21 to 23% by weight urea agent and 10% to 80% by weight of polyol. In certain embodiments, the first phase includes about 23 to 25% by weight urea agent and a non-aqueous solvent selected from the group consisting of: C(₂₋₆) alkanediols, glycol ethers, dimethyl ethers.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of polyether polyol. In some embodiments, the first phase includes 1-5%, 5-10%, 10-15%, or 15-20% by weight of a urea agent, and 10% to 80% by weight of polyether polyol. In certain embodiments, the first phase includes about 5% by weight of a urea agent and 10% to 80% by weight of polyether polyol. In certain embodiments, the first phase includes about 15% by weight of a urea agent and 10% to 80% by weight of polyether polyol. In certain embodiments, the first phase includes about 20% by weight of a urea agent and 10% to 80% by weight of polyether polyol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of polyester polyol. In some embodiments, the first phase includes 1-5%, 5-10%, 10-15%, or 15-20% by weight of a urea agent, and 10% to 80% by weight of polyester polyol. In certain embodiments, the first phase includes about 5% by weight of a urea agent and 10% to 80% by weight of polyester polyol. In certain embodiments, the first phase includes about 15% by weight of a urea agent and 10% to 80% by weight of polyester polyol. In certain embodiments, the first phase includes about 20% by weight of a urea agent and 10% to 80% by weight of polyester polyol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of glycerol (1,2,3-propanetriol). In some embodiments, the first phase includes 1-5%, 5-10%, 10-15%, or 15-20% by weight of a urea agent, and 10% to 80% by weight of glycerol (1,2,3-propanetriol). In certain embodiments, the first phase includes about 5% by weight of a urea agent and 10% to 80% by weight of glycerol (1,2,3-propanetriol). In certain embodiments, the first phase includes about 15% by weight of a urea agent and 10% to 80% by weight of glycerol (1,2,3-propanetriol). In certain embodiments, the first phase includes about 20% by weight of a urea agent and 10% to 80% by weight of glycerol (1,2,3-propanetriol).

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of diglycerol. In some embodiments, the first phase includes 1-5%, 5-10%, 10-15%, or 15-20% by weight of a urea agent, and 10% to 80% by weight of diglycerol. In certain embodiments, the first phase includes about 5% by weight of a urea agent and 10% to 80% by weight of diglycerol. In certain embodiments, the first phase includes about 15% by weight of a urea agent and 10% to 80% by weight of diglycerol. In certain embodiments, the first phase includes about 20% by weight of a urea agent and 10% to 80% by weight of diglycerol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of propylene glycol (1,2-propanediol). In some embodiments, the first phase includes 1-5%, 5-10%, 10-15%, or 15-20% by weight of a urea agent, and 10% to 80% by weight of propylene glycol (1,2-propanediol). In certain embodiments, the first phase includes about 5% by weight of a urea agent and 10% to 80% by weight of propylene glycol (1,2-propanediol). In certain embodiments, the first phase includes about 15% by weight of a urea agent and 10% to 80% by weight of propylene glycol (1,2-propanediol). In certain embodiments, the first phase includes about 20% by weight of a urea agent and 10% to 80% by weight of propylene glycol (1,2-propanediol).

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of dipropylene glycol. In some embodiments, the first phase includes 1-5%, 5-10%, 10-15%, or 15-20% by weight of a urea agent, and 10% to 80% by weight of dipropylene glycol. In certain embodiments, the first phase includes about 5% by weight of a urea agent and 10% to 80% by weight of dipropylene glycol. In certain embodiments, the first phase includes about 15% by weight of a urea agent and 10% to 80% by weight of dipropylene glycol. In certain embodiments, the first phase includes about 20% by weight of a urea agent and 10% to 80% by weight of dipropylene glycol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,3-propanediol. In some embodiments, the first phase includes 1-5%, 5-10%, 10-15%, or 15-20% by weight of a urea agent, and 10% to 80% by weight of 1,3-propanediol. In certain embodiments, the first phase includes about 5% by weight of a urea agent and 10% to 80% by weight of 1,3-propanediol. In certain embodiments, the first phase includes about 15% by weight of a urea agent and 10% to 80% by weight of 1,3-propanediol. In certain embodiments, the first phase includes about 20% by weight of a urea agent and 10% to 80% by weight of 1,3-propanediol. In some embodiments, the first phase includes about 5 to 7% by weight urea agent and 1,3-propanediol. In some embodiments, the first phase includes about 7 to 9% by weight urea agent and 1,3-propanediol. In certain embodiments, the first phase includes about 9 to 11% by weight urea agent and 1,3-propanediol. In certain embodiments, the first phase includes about 11 to 13% by weight urea agent and 1,3-propanediol. In certain embodiments, the first phase includes about 13 to 15% by weight urea agent and 1,3-propanediol. In certain embodiments, the first phase includes about 15 to 17% by weight urea agent and 1,3-propanediol. In certain embodiments, the first phase includes about 17 to 19% by weight urea agent and 1,3-propanediol. In certain embodiments, the first phase includes about 19 to 21% by weight urea agent and 1,3-propanediol. In certain embodiments, the first phase includes about 21 to 23% by weight urea agent and 1,3-propanediol. In certain embodiments, the first phase includes about 23 to 25% by weight urea agent and 1,3-propanediol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of butylene glycol (1,3-butanediol). In some embodiments, the first phase includes 1-5%, 5-10%, 10-15%, or 15-20% by weight of a urea agent, and 10% to 80% by weight of butylene glycol (1,3-butanediol). In certain embodiments, the first phase includes about 5% by weight of a urea agent and 10% to 80% by weight of butylene glycol (1,3-butanediol). In certain embodiments, the first phase includes about 15% by weight of a urea agent and 10% to 80% by weight of butylene glycol (1,3-butanediol). In certain embodiments, the first phase includes about 20% by weight of a urea agent and 10% to 80% by weight of butylene glycol (1,3-butanediol).

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2-butanediol. In some embodiments, the first phase includes 1-5%, 5-10%, 10-15%, or 15-20% by weight of a urea agent, and 10% to 80% by weight of 1,2-butanediol. In certain embodiments, the first phase includes about 5% by weight of a urea agent and 10% to 80% by weight of 1,2-butanediol. In certain embodiments, the first phase includes about 15% by weight of a urea agent and 10% to 80% by weight of 1,2-butanediol. In certain embodiments, the first phase includes about 20% by weight of a urea agent and 10% to 80% by weight of 1,2-butanediol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of pentylene glycol (1,2-pentanediol). In some embodiments, the first phase includes 1-5%, 5-10%, 10-15%, or 15-20% by weight of a urea agent, and 10% to 80% by weight of pentylene glycol (1,2-pentanediol). In certain embodiments, the first phase includes about 5% by weight of a urea agent and 10% to 80% by weight of pentylene glycol (1,2-pentanediol). In certain embodiments, the first phase includes about 15% by weight of a urea agent and 10% to 80% by weight of pentylene glycol (1,2-pentanediol). In certain embodiments, the first phase includes about 20% by weight of a urea agent and 10% to 80% by weight of pentylene glycol (1,2-pentanediol).

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,5-pentanediol. In some embodiments, the first phase includes 1-5%, 5-10%, 10-15%, or 15-20% by weight of a urea agent, and 10% to 80% by weight of 1,5-pentanediol. In certain embodiments, the first phase includes about 5% by weight of a urea agent and 10% to 80% by weight of 1,5-pentanediol. In certain embodiments, the first phase includes about 15% by weight of a urea agent and 10% to 80% by weight of 1,5-pentanediol. In certain embodiments, the first phase includes about 20% by weight of a urea agent and 10% to 80% by weight of 1,5-pentanediol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2-hexanediol. In some embodiments, the first phase includes 1-5%, 5-10%, 10-15%, or 15-20% by weight of a urea agent, and 10% to 80% by weight of 1,2-hexanediol. In certain embodiments, the first phase includes about 5% by weight of a urea agent and 10% to 80% by weight of 1,2-hexanediol. In certain embodiments, the first phase includes about 15% by weight of a urea agent and 10% to 80% by weight of 1,2-hexanediol. In certain embodiments, the first phase includes about 20% by weight of a urea agent and 10% to 80% by weight of 1,2-hexanediol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,6-hexanediol. In some embodiments, the first phase includes 1-5%, 5-10%, 10-15%, or 15-20% by weight of a urea agent, and 10% to 80% by weight of 1,6-hexanediol. In certain embodiments, the first phase includes about 5% by weight of a urea agent and 10% to 80% by weight of 1,6-hexanediol. In certain embodiments, the first phase includes about 15% by weight of a urea agent and 10% to 80% by weight of 1,6-hexanediol. In certain embodiments, the first phase includes about 20% by weight of a urea agent and 10% to 80% by weight of 1,6-hexanediol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2,3-hexanetriol. In some embodiments, the first phase includes 1-5%, 5-10%, 10-15%, or 15-20% by weight of a urea agent, and 10% to 80% by weight of 1,2,3-hexanetriol. In certain embodiments, the first phase includes about 5% by weight of a urea agent and 10% to 80% by weight of 1,2,3-hexanetriol. In certain embodiments, the first phase includes about 15% by weight of a urea agent and 10% to 80% by weight of 1,2,3-hexanetriol. In certain embodiments, the first phase includes about 20% by weight of a urea agent and 10% to 80% by weight of 1,2,3-hexanetriol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2,6-hexanetriol. In some embodiments, the first phase includes 1-5%, 5-10%, 10-15%, or 15-20% by weight of a urea agent, and 10% to 80% by weight of 1,2,6-hexanetriol. In certain embodiments, the first phase includes about 5% by weight of a urea agent and 10% to 80% by weight of 1,2,6-hexanetriol. In certain embodiments, the first phase includes about 15% by weight of a urea agent and 10% to 80% by weight of 1,2,6-hexanetriol. In certain embodiments, the first phase includes about 20% by weight of a urea agent and 10% to 80% by weight of 1,2,6-hexanetriol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of ethoxydiglycol. In some embodiments, the first phase includes 1-5%, 5-10%, 10-15%, or 15-20% by weight of a urea agent, and 10% to 80% by weight of ethoxydiglycol. In certain embodiments, the first phase includes about 5% by weight of a urea agent and 10% to 80% by weight of ethoxydiglycol. In certain embodiments, the first phase includes about 15% by weight of a urea agent and 10% to 80% by weight of ethoxydiglycol. In certain embodiments, the first phase includes about 20% by weight of a urea agent and 10% to 80% by weight of ethoxydiglycol.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of dimethyl isosorbide. In some embodiments, the first phase includes 1-5%, 5-10%, 10-15%, or 15-20% by weight of a urea agent, and 10% to 80% by weight of dimethyl isosorbide. In certain embodiments, the first phase includes about 5% by weight of a urea agent and 10% to 80% by weight of dimethyl isosorbide. In certain embodiments, the first phase includes about 15% by weight of a urea agent and 10% to 80% by weight of dimethyl isosorbide. In certain embodiments, the first phase includes about 20% by weight of a urea agent and 10% to 80% by weight of dimethyl isosorbide.

In some embodiments, the first phase includes about 1 to 30% by weight of a urea agent e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of glycol ether, dimethyl ether, or a combination thereof. In some embodiments, the first phase includes 1-5%, 5-10%, 10-15%, or 15-20% by weight of a urea agent, and 10% to 80% by weight of glycol ether, dimethyl ether, or a combination thereof. In certain embodiments, the first phase includes about 5% by weight of a urea agent and 10% to 80% by weight of glycol ether, dimethyl ether, or a combination thereof. In certain embodiments, the first phase includes about 15% by weight of a urea agent and 10% to 80% by weight of glycol ether, dimethyl ether, or a combination thereof. In certain embodiments, the first phase includes about 20% by weight of a urea agent and 10% to 80% by weight of glycol ether, dimethyl ether, or a combination thereof.

In some embodiments, the subject composition comprises a second phase comprising the homogenous first phase solution and azelaic acid. In some embodiments, the second phase comprises the first phase solution and about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight of azelaic acid.

### Additional Components

A formulation may contain one or more (optional) additional ingredients. Any convenient ingredient known to the skilled artisan to provide cosmetic/aesthetic benefits can be utilized in the subject formulations. Such cosmetic/aesthetic benefits include, but are not limited to, reducing the appearance of fine lines/wrinkles, improving skin barrier function (by reducing the rate/extent of trans-epidermal water loss), making the skin feel smoother/more supple/softer, creating the appearance of more even skin tone (reducing dyschromia) and/or “glow”/radiance (also described in the art as “brightness”).

In some embodiments, the composition further includes one or more optional additional components (e.g., as described herein). In some embodiments, the one or more optional additional components are added in the first phase solution. In some embodiments, the one or more optional additional components are added in the second phase solution.

In some embodiments, the one or more optional additional components are selected from tocopherols, tocotrienols (e.g., alpha, beta, delta and gamma tocopherols or alpha, beta, delta and gamma tocotrienols), ferulic acid, azelaic acid, hydroxy acids (e.g., salicylic acid), panthenol, Pinus pinaster bark extract, emulsifying agent, hyaluronic acid complex, madecassoside, acetyl zingerone, bakuchiol, and bis-ethylhexyl hydroxydimethoxy benzylmalonate.

In some embodiments, the optional additional component is salicylic acid. In some embodiments, the amount of salicylic acid in the subject composition ranges from 0.1% to 5% by weight of salicylic acid, such as 0.1%, 0.2%, 0.3%, 0.4%, 0.5%, 0.6%, 0.7%, 0.8%, 0.9%, 1%, 2% to 3%, 3% to 4%, 4% to 5%, 2% to 5%, by weight of salicylic acid.

Each optional additional component (e.g., as described herein) may be present in an amount of 15% or less by weight of the composition, such as 14% or less, 13% or less, 12% or less, 11% or less, 10% or less, 9% or less, 8% or less, 7% or less, 6% or less, 5% or less, 4% or less, 3% or less, 2% or less, 1% or less by weight. In some embodiments the total amount of the one or more optional additional components (e.g., as described herein) in the composition 10% or less by weight, such as 9% or less, 8% or less, 7% or less, 6% or less, 5% or less, 4% or less, 3% or less, 2% or less, 1% or less by weight.

In some embodiments, the composition further includes 10% or less by weight in total of one or more optional additional components selected from an antioxidant, a skin lightening agent, and a moisturizing agent.

### Vitamin C Agent

In some embodiments, the composition further includes an optional additional component that is vitamin C. In some embodiments, the vitamin C agent is in the form selected from ascorbic acid, 3-O ethyl ascorbic acid, magnesium ascorbyl phosphate, sodium ascorbyl phosphate, tetrahexyldecyl ascorbate, and combinations thereof. In some embodiments, the vitamin C agent is ascorbic acid.

The terms “ascorbic acid”, “L-ascorbic acid” and “vitamin C” are used interchangeably herein, and refer to the naturally occurring vitamin of CAS Registry Number: 50-81-7. Any convenient form of ascorbic acid can be utilized in the subject formulations. In some embodiments, the ascorbic acid used of the present disclosure is a powder.

In certain embodiments, the ascorbic acid material used in preparing the subject compositions is composed of granular particles. Such a particulate powder has a particle size (e.g., mean particle size) of less than about 25 microns, such as less than about 20 microns, and less than about 12.5 microns, e.g., as measured by a Hagman gauge. In some embodiments, all of the ascorbic acid powder used in preparing the subject compositions is capable of passage through a No. 100 U.S. Standard Sieve, a standard testing procedure used by the US Pharmacopoeia. In some embodiments, 80% or more (such as 90% or more, or 100%) of ascorbic acid powder used in preparing the subject composition is capable of passage through a No. 325 U.S. Standard Sieve. For example, one powder meeting the above criterion is Ascorbic Acid Ultra-Fine Powder from DSM Nutritional Products LLC, Parsippany, NJ. Previously, this product was available as Product Code No. 6045653 from Roche Vitamins and Fine Chemicals.

In some embodiments, the amount of ascorbic acid in the subject composition is at least about 5% by weight, such as at least about 10% by weight, at least about 12% by weight, at least about 15% by weight, at least about 20% by weight.

In some embodiments, the amount of ascorbic acid in the subject composition is at least about 5% by weight, such as at least about 10% by weight, at least about 12% by weight, at least about 15% by weight, at least about 20% by weight, or at least about 25% by weight. In some embodiments, the subject composition includes about 28% by weight or less of ascorbic acid in the non-aqueous solvent solution, such as about 25% by weight or less. In certain embodiments, the non-aqueous solvent is 1,3-propanediol. In particular embodiments, the amount of ascorbic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of ascorbic acid in the subject composition is about 5%, about 10%, about 15%, about 20%, or about 25% by weight.

In some embodiments, the amount of ascorbic acid in the subject composition is about 5%, about 6%, about 7%, about 8%, about 9%, about 10%, about 11%, about 12%, about 13%, about 14%, about 16%, about 17%, about 18%, about 19%, about 20%, about 21%, about 22%, about 23%, about 24%, about 25%, about 26%, about 27%, about 28%, about 29%, or about 30% by weight.

In some embodiments, the vitamin C agent is present in the composition in an amount of 15% or less by weight, such as 10% or less, 5% or less by weight. In some embodiments, the vitamin C agent is absent from the composition.

### Tocopherol or Tocotrienol Agent

In some embodiments, the composition further includes an optional additional component that is a tocopherol or tocotrienol agent. In some embodiments, the tocopherol or tocotrienol agent is a form of Vitamin E selected from alpha, beta, delta and gamma tocopherols and alpha, beta, delta and gamma tocotrienols, and combinations thereof. In some embodiments, the tocopherol or tocotrienol is alpha-tocopherol.

In some embodiments, the tocopherol or tocotrienol agent is present in the composition in an amount of 2% or less by weight, such as 1.5% or less, 1% or less, or 0.5% or less by weight.

In some embodiments of any one of the formulations described herein, the formulation excludes tocopherol or tocotrienol agents, e.g., or precursors thereof having vitamin E activity. In certain embodiments of any one of the formulations described herein, the formulation excludes vitamin E acetate.

### Antioxidants

In some embodiments, the one or more additional components is an antioxidant.

In certain embodiments, the formulation contains a secondary antioxidant (i.e., in addition to the optional additive Vitamin C or the optional additive tocopherol or tocotrienol agent), or a primary antioxidant (i.e., without the optional vitamin C or optional additive tocopherol or tocotrienol agent).

Preferred antioxidants include cinnamic acid derivatives (e.g., ferulic acid, caffeic acid, or coumaric acid), terpenoid antioxidants, and benzoic acid derivatives (e.g., p-hydroxy benzoic acid, gallic acid, or protocatechuic acid). Pinus pinaster Bark/Bud Extract (available under the tradename Pycnogenol® from DKSH North America, Inc., or from Res Pharma Industriale under the tradename Pantrofina® Skin360) contains these cinnamic acid derivatives and benzoic acid derivatives, and is, therefore, a preferred primary or secondary antioxidant.

In some embodiments, the primary or secondary antioxidant is zingerone or acetyl zingerone. In some embodiments, the primary or secondary antioxidant is bakuchiol (10309-37-2) a natural terpenoid antioxidant. In some embodiments, the secondary antioxidant is bis-ethylhexyl hydroxydimethoxy benzylmalonate (HDBM).

In some embodiments, the primary or secondary antioxidant is an antioxidant blend comprising Vitis vinifera (Grape) Seed Extract, Camellia sinensis Leaf Extract, Quercus robur Wood Extract, Pinus pinaster Bark Extract.

In some embodiments, the primary or secondary antioxidant is acetyl zingerone.

The primary or secondary antioxidant, when included, is preferably present in an amount in the range of 0.1 to 5%, 0.1 to 3%, more preferably 0.1 to 2% by weight of the composition, such as 0.1 to 1% by weight, 0.1 to 0.5% by weight, e.g., about 0.2%, about 0.3%, about 0.4% or about 0.5% by weight.

### Skin Lightening Agents

In certain embodiments, the formulation contains a secondary skin lightening agent (e.g., as defined herein). Skin lightening agents which may be included in compositions of the present disclosure include, but are not limited to: hydroquinone and its derivatives, including, for example, its monomethyl and monobenzyl ethers; licorice root (Glycyrrhiza glabra) extract; kojic acid; arbutin; retinoids (including all-trans-retinoic acid, adapalene and tazarotene); alpha hydroxy acids, in particular citric acid, lactic acid, and glycolic acid; ellagic acid; gluconic acid; gentisic acid (2,5-dihydrobenzoic acid); 4-hydroxy benzoic acid; salts and esters of the above-mentioned acids, including ammonium lactate and sodium lactate; N-acetyl glucosamine; aloesin, a hydroxymethyl chromone isolated from aloe vera; Vitamin B3 compound or its derivative—niacin, nicotinic acid, niacinamide. Epigallocatechin 3-O-gallate (EGCG), and other catechin constituents of tea extracts, in particular green tea; extract of soybean oil (Glycine soja), including isoflavones; hydroxystilbene; butyl hydroxy anisole; and butyl hydroxy toluene may also be utilized as a skin lightening agent. In some embodiments, the additional skin lightening agent is azelaic acid or arbutin.

The skin lightening agent, when included, is preferably present in an amount in the range of 0.1 to 10%, more preferably 0.2 to 5% by weight of the composition, such as 0.2 to 4% by weight, 0.2 to 3% by weight, or 0.2 to 2% by weight. In certain embodiments, the secondary skin lightening agent is soluble and may be added directly to the azelaic acid concentrate of the present invention. The secondary skin lightening agent may also be encapsulated using techniques known to the person having ordinary skill in the art.

### Hydroxy Acids

In some embodiments, formulation contains a hydroxy acid, e.g., a small molecule compound including a carboxylic acid and a hydroxy group. The acid may be an alkyl carboxylic acid or a benzoic acid. The hydroxy group can be a phenol or an alkyl alcohol. In certain embodiments, the hydroxy acid is an alpha-hydroxy carboxylic acid. In certain embodiments the hydroxy acid contains 2-12 carbon atoms, such as 2-6 or 2-4 carbons. Hydroxy acids of interest include, but are not limited to, glycolic acid, lactic acid, mandelic acid, salicylic acid, capryloyl salicylic acid, salicyloyl phytosphingosine, gluconolactone, lactobionic acid, maltobionic acid, and combinations thereof.

### Anti-Inflammatory

In some embodiments, formulation contains an anti-inflammatory agent as an additional ingredient. In some embodiments, the anti-inflammatory agent is madecassoside, madecassoside asiaticoside, or madecassic acid. The anti-inflammatory agent, when included, is preferably present in an amount in the range of 0.1 to 2%, more preferably 0.1 to 1% by weight of the composition, such as 0.1 to 0.5% by weight, or 0.1 to 0.2% by weight. In some embodiments, madecassoside or madecassoside asiaticoside, is included in an amount in the range of 0.1 to 0.5%, such as about 0.1% or about 0.2% by weight.

### Exemplary Topical Formulations

In some embodiments, the topical composition includes: a) 4% to 12% by weight azelaic acid; and b) 5% to 20% by weight of a urea agent, dissolved in a non-aqueous skin-compatible solvent selected from polyol, C(2-6) alkanediol, glycol ether, dimethyl ether, or a combination thereof.

In some embodiments, the formulations of the present disclosure are concentrates which are generally: free of silicones, and “substantially free” of water, and “substantially free” of volatile alcohols or monohydric alcohols. By “substantially free” of water is meant that (i) water is not intentionally added to the concentrate, and (ii) the amount of water in the concentrate is less than about 2% by weight of the concentrate, preferably less than 1% by weight, more preferably less than about 0.5%, and still more preferably less than about 0.1%. In some embodiments, water is absent and the composition can be referred to as anhydrous. For example, water has a negative effect on the stability of urea concentration of the composition, promoting breakdown of urea within the composition. Therefore, the composition is substantially free of water, and preferably is anhydrous, and provides for an increase in the stability of urea and prevents breakdown or degradation of urea within the composition. In such cases, the composition may be referred to as non-aqueous.

By “substantially free” of volatile alcohols, such as monohydric alcohols, is meant that (i) volatile alcohols are not intentionally added to the concentrate, and (ii) the amount of volatile alcohols in the concentrate is less than about 2% by weight of the concentrate, preferably less than 1% by weight, more preferably less than about 0.5%, and still more preferably less than about 0.1%. In some embodiments, the volatile alcohols are absent. Similar to the effect of water on urea, volatile alcohols such as monohydric alcohols, have a negative effect on the stability of azelaic acid, promoting breakdown of azelaic acid within a composition. Therefore, the composition is substantially free of volatile alcohols and to increase the stability of azelaic acid and prevent breakdown of azelaic acid within the composition.

In certain embodiments, the concentrate is also substantially free of oils and/or lipids. In such cases, the composition may be referred to as an oil and lipid free composition.

### Emulsion Agents

It is understood that any of the non-aqueous liquid compositions having particular amounts of azelaic acid (e.g., as described herein) can be combined with an immiscible phase or ingredient (e.g., an oil component) to produce an emulsion composition. In some embodiments, the non-aqueous liquid composition that makes up the first phase of an emulsion composition is referred to as a concentrate. The liquid concentrate can be mixed with one or more additional components (e.g., an immiscible oil phase or component and an optional emulsifying agent) to produce an emulsion. A variety of methods and ingredients for preparing emulsions are available and can be used in the subject emulsion compositions.

In some embodiments, an emulsion composition of this disclosure is referred to as a gel.

Any convenient oils and lipids can be utilized in the oil component of the subject emulsions. An oil component or oil phase refers to any phase that is immiscible with the non-aqueous liquid composition. In some embodiments, the oil component is silicone-based, e.g., includes a silicone polymer. In some embodiments, the oil component includes a silicone oil or silicone elastomer, such as a polyorganosiloxane. In some embodiments, the silicone polymers have dual characteristics, and can be used as emulsifiers and/or act as the continuous/dispersed phase of the emulsion composition.

Oils and lipids of interest include, but are not limited to, silicone oils, linseed oil, tsubaki oil, macadamia nut oil, corn oil, mink oil, olive oil, avocado oil, sasanqua oil, castor oil, safflower oil, apricot oil, cinnamon oil, jojoba oil, grape oil, sunflower oil, almond oil, rapeseed oil, sesame oil, wheat germ oil, rice germ oil, rice bran oil, cottonseed oil, soybean oil, peanut oil, teaseed oil, evening primrose oil, eggyoke oil, neetsfoot oil, liver oil, triglycerine, glycerine trioctanate, pentaerythritol tetraoctanate, glycerine triisopalmitate, cholesterol, free fatty acids, and combinations thereof.

Any convenient emulsifying agents or emulsifiers can be utilized in the preparation of the subject emulsions to stabilize the composition and prevent separation of the oil component from the solvent solution (e.g., the non-aqueous liquid composition). Exemplary emulsifying agents include but are not limited to polysorbates, laureth-4, potassium cetyl sulfate, and silicone and silicone-elastomer-based emulsifiers and emulsifying blends. In some embodiment, a surfactant such as a monoglyceride, sorbitan fatty acid ester, or polyglycerine fatty acid ester, polyoxyethylene hardened castor oil, polyoxyethylene fatty acid ether, is added thereto, e.g. 5% by weight or more, and the stability is further improved.

In some embodiments, the formulations of the present disclosure contain about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of an emulsifying agent. In some embodiments, the formulations of the present disclosure contain an emulsifying agent ranging from about 4% to about 6%, about 6% to about 8%, about 8% to about 10%, about 10% to about 12% about 14%, about 14% to about 16%, about 16% to about 18%, about 18% to about 20%, about 20% to about 22%, about 22% to about 24%, about 24% to about 26%, about 26% to about 28%, or about 28% to about 30% by weight.

In some embodiments, the emulsifying agent is a silicone-compatible agent.

In some embodiments, emulsifying agents include, but are not limited to, polysorbates, laureth-4, potassium cetyl sulfate, and silicone and silicone-elastomer-based emulsifiers and emulsifying blends. In some embodiments, a surfactant such as a monoglyceride, sorbitan fatty acid ester, or polyglycerine fatty acid ester, polyoxyethylene hardened castor oil, polyoxyethylene fatty acid ether, or combinations thereof, is added thereto in a small amount, and the stability is further improved. In some embodiments, the emulsifying agent is added to the formulation in an effective amount to improve the stability of the formulation.

In some embodiments, the emulsifying agents include, but are not limited to: sorbitan laurate, sorbitan palmitate, sorbitan sesquiisostearate, sorbitan sesquioleate, sorbitan sesquistearate, sorbitan stearate, sorbitan oleate, sorbitan monoisostearate, sorbitan trisostearate, sorbitan trioleate, sorbitan tristearate; glyceryl behenate, glyceryl caprate, glyceryl caprylate, glyceryl caprylate/caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate; polyglyceryl-4 isostearate, polyglyceryl-3 oleate, polyglyceryl-2 sesquioleate, triglyceryl diisostearate, diglyceryl monooleate, tetraglyceryl monooleate, glycol distearate, glycol hydroxystearate, glycol oleate, glycol ricinoleate, glycol stearate, propylene glycol isostearate, propylene glycol hydroxystearate, propylene glycol laurate, propylene glycol myristate, propylene glycol oleate, propylene glycol ricinoleate, propylene glycol stearate; sucrose cocoate, sucrose laurate; Methyl Glucose Sesquistearate, Methyl Glucose Dioleate; PEG-20 Methyl Glucose Sesquistearate; or mixtures thereof.

### Silicone Agents

The compositions of this disclosure can in some cases be formulated as an emulsion, e.g., by combining a first phase solution of a non-aqueous skin-compatible solvent with a second phase including an emulsifying agent. In some embodiments, the emulsifying agent of certain formulations of the present disclosure contain one or more silicone emulsifiers (e.g. silicone agents). In some embodiments, the homogenous solution containing azelaic acid, a urea agent, and a non-aqueous solvent (e.g. the non-aqueous liquid composition) is mixed with a second phase solution containing one or more silicone emulsifying agents to form an emulsion formulation. In some embodiments, a first phase solution containing a urea agent and a non-aqueous solvent (e.g. the non-aqueous liquid composition) is mixed with a second phase solution containing azelaic acid; and the second phase solution containing azelaic acid, a urea agent, and a non-aqueous solvent is mixed with one or more silicone emulsifying agents to form an emulsion formulation. In some embodiments, the emulsion prevents degradation of the urea agent and/or azelaic acid. In some embodiments, the emulsion prevents precipitation of the urea agent and/or azelaic acid. In some embodiments, the emulsion prevents oxygen or air from degrading the urea agent and/or azelaic acid within the emulsion. In some embodiments, the emulsion prevents moisture from degrading the urea agent and/or azelaic acid within the emulsion. In some embodiments, the emulsion prevents absorption of water from degrading the urea agent and/or azelaic acid within the emulsion.

In some embodiments, the emulsion increases the shelf life of the emulsion containing the urea agent. In some embodiments, the shelf life of the emulsion ranges from 6 weeks to 8 weeks, 2 months 4 months, 4 months to 6 months, 6 months to 1 year, 1 to 1.5 years, 1.5 to 2 years, 2 to 2.5 years, 2.5 to 3 years, 3 to 3.5 years, or 3.5 to 4 years. In some embodiments, the shelf life is about 1 month or more, about 2 months or more, about 3 months or more, about 4 months or more, about 5 months or more, about 6 months or more, about 1 year or more, about 1.5 years or more, about 2 years or more, or about 2.5 years or more.

Silicone agents of interest include, but are not limited to, dimethicone, adimethicone, cyclopentasiloxane, dimethicone/PEG-10/15 crosspolymer, lauryl PEG-9 polydimethylsiloxyethyl dimethicone, PEG-3 dimethicone, PEG-10 dimethicone, PEG-9 methyl ether dimethicone, polyglyceryl-3 polydimethylsiloxyethyl dimethicone, PEG/PPG-18/18 dimethicone, PEG-15/lauryl dimethicone crosspolymer, PEG-15/lauryl polydimethylsiloxyethyl dimethicone crosspolymer, dimethicone/polyglycerin-3 crosspolymer, and dimethicone/vinyl eimethicone crosspolymer.

In some embodiments, the formulations of the present disclosure contain about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight of one or more silicone agents. In some embodiments, the formulations of the present disclosure contain an amount of one or more silicone agents ranging from about 1 to 5%, 5 to 10%, 10 to 15%, 15 to 20%, 20 to 25%, or 25 to 30% by weight.

In some embodiments, the composition of the present disclosure includes an external phase (e.g., emulsion phase) comprising a silicone agent. In some embodiments, the azelaic acid and urea agent dissolved in the non-aqueous solvent is suspended in the external phase. In some embodiments, the phase contains about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight of one or more silicone agents. In some embodiments, the external phase contains an amount of one or more silicone agents ranging from about 1 to 10%, 10 to 20%, 20 to 30%, 30 to 40%, 40 to 50%, 50 to 60%, 60 to 70%, 70 to 80%, 80 to 90%, or 90 to 100% by weight. In some embodiments, the external phase contains an amount of one or more silicone agents ranging from about 1 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 80 to 85%, 85 to 90%, 90 to 95%, or 95 to 100% by weight.

In some embodiments, the external phase comprises, in addition to one or more silicone agents, a non-silicone oil agent. In some embodiments, the external phase comprises, in addition to one or more silicone agents, 0.5 to 1%, 1 to 1.5%, 1.5 to 2%, 2 to 2.5%, 2.5 to 3%, 3 to 3.5%, 3.5 to 4%, 4 to 4.5%, 4.5 to 5%, 5 to 5.5%, 6 to 6.5%, 6.5 to 7%, 7 to 7.5%, 8 to 8.5%, 8.5% to 9%, 9 to 9.5%, or 9.5 to 10% by weight of a non-silicone oil agent. In some embodiments, the external phase contains an amount of one or more silicone agents ranging from about 1 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 80 to 85%, 85 to 90%, 90 to 95%, or 95 to 100% by weight; and 0.5 to 1%, 1 to 1.5%, 1.5 to 2%, 2 to 2.5%, 2.5 to 3%, 3 to 3.5%, 3.5 to 4%, 4 to 4.5%, 4.5 to 5%, 5 to 5.5%, 6 to 6.5%, 6.5 to 7%, 7 to 7.5%, 8 to 8.5%, 8.5% to 9%, 9 to 9.5%, or 9.5 to 10% by weight of a non-silicone oil agent.

In some embodiments, the silicone agent is dimethicone. In some embodiments, the silicone agent contains dimethicone in combination with a dimethicone/PEG-10/15 crosspolymer. In some embodiments, the silicone agent contains dimethicone in combination with a dimethicone/PEG-10/15 crosspolymer and lauryl PEG-9 polydimethylsiloxyethyl

dimethicone. In some embodiments, the silicone agent contains about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% dimethicone alone or in combination with a dimethicone/PEG-10/15 crosspolymer and/or lauryl PEG-9 polydimethylsiloxyethyl. In some embodiments, the silicone agent contains about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% dimethicone; about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% dimethicone/PEG-10/15 crosspolymer; and/or about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% lauryl PEG-9 polydimethylsiloxyethyl.

In some embodiments, the subject composition includes a ratio of the second phase (e.g., homogenous solution containing azelaic acid and the urea agent) to the emulsion phase. In certain embodiments, the ratio of the second phase to the emulsion phase is 1 to 5, such as a ratio of 1.0 (i.e., 1:1), 1.25, 1.50, 1.75, 2.0 (i.e. 2:1), 2.25, 2.50, 2.75, 3.0, 3.25, 3.50, 3.75, 4.0, 4.25, 4.50, 4.75, or 5. In certain embodiments, the ratio of the second phase to the emulsion phase is 1.8 to 2.2, such as a ratio of 2. In certain embodiments, the ratio of the second phase to the emulsion phase is 1.0 to 1.3, such as a ratio of 1.25 or a ratio of 1.0. In certain embodiments, the ratio of the second phase to the emulsion phase ranges from 1 to 20, such as a ratio of 1 (i.e., 1:1), 2, 3, 4, 5, 6, 7, 8, 9, 10, 11, 12, 13, 14, 15, 16, 17, 18, 19, or 20. In certain embodiments, the ratio of the second phase to the emulsion phase is 1.8 to 2.2, such as a ratio of 2. In certain embodiments, the ratio of the second phase to the emulsion phase is 1.0 to 1.3, such as a ratio of 1.25 or a ratio of 1.0. In some embodiments, the ratio of the second phase to the emulsion phase is up to 19 (e.g., 19:1). In some embodiments, the ratio of the second phase to the emulsion phase ranges from 1 to 19. In some embodiments, the ratio of the second phase to the emulsion phase is 9 (i.e., 9:1). In some embodiments, the ratio of the second phase to the emulsion phase is 19 (i.e., 19:1).

### Containers

Any containers suitable for storing and/or dispensing the subject formulations can be adapted for use. The container can provide a sealed environment for containing the composition, and separation from the atmosphere. The container can prevent during storage undesirable degradation, e.g., from absorption of light and/or moisture from the atmosphere or surrounding environment. Provided are ready-to-use topical preparations of azelaic acid in a multi-use container which is pre-filled with a storage stable topical composition (e.g., as described herein).

Additional packaging for the container can be included. In some cases, the packaging provides a further barrier that prevents absorption of light and/or moisture from the atmosphere or surrounding environment.

### Methods of Preparation

Also provided by this disclosure are processes for solubilizing azelaic acid for that include preparation of any one of the subject formulations (e.g., as described herein), e.g., by dissolving azelaic acid in a non-aqueous solvent with a urea agent and one or more optionally additional components to provide a completely solubilized liquid composition.

In some embodiments, the process includes combining:
1. 1% to 20% by weight urea agent selected from urea, hydroxyethyl urea, and combination thereof;
2. 10% to 94% by weight of a non-aqueous skin-compatible solvent comprising C₍₃₋₆₎polyol, ethoxydiglycol, dimethyl ether, or a combination thereof; and
3. optionally one or more additional agents; with
4. 4% to 12% by weight azelaic acid;
   thereby dissolving the azelaic acid to produce nonaqueous, single-phase clear liquid composition of azelaic acid. In certain embodiments, the one or more additional agents are combined and include: 0.1% to 2% madecassoside or madecassoside asiaticoside, 0.1-2% asiaticoside; and 0.5% to 2% pinus pinaster bark extract. In certain embodiments, the one or more additional agents are combined and include: 3% to 10% by weight azelaic acid.

In some embodiments, the process further includes: combining 0.5% to 2% by weight of Vitamin E and 1.5% to 5% by weight of an emulsifying agent to produce a second liquid composition; and combining the second liquid composition with the liquid composition of azelaic acid to produce an emulsion. In some embodiments, the process further includes: combining 0.5% to 2% by weight of a lipid component and 1.5% to 5% by weight of an emulsifying agent to produce a second liquid composition; and combining the second liquid composition with the liquid composition of azelaic acid to produce an emulsion.

In some embodiments of the process, the one or more additional agents are combined and include: 0.5% to 2% by weight hydroxy acid. In certain embodiments, the hydroxy acid is selected from glycolic acid, lactic acid, mandelic acid, salicylic acid, capryloyl salicylic acid, salicyloyl phytosphingosine, gluconolactone, lactobionic acid, maltobionic acid, and combinations thereof.

Also provided are product storage stable formulations produced by the process according to any one of the embodiments described herein.

### Definitions

The following definitions are set forth to illustrate and define the meaning and scope of the terms used in the description.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

“At least one” means one or more, and also includes individual components as well as mixtures/combinations.

Numbers used in describing quantities of ingredients and/or reaction conditions are to be understood as being modified in all instances by the term “about.” Unless otherwise indicated, percentages and ratios are to be understood as based upon the total weight of the concentrate.

Numerical ranges are meant to include numbers within the recited range, and combinations of subranges between the given ranges. For example, a range from 1-5 includes 1, 2, 3, 4 and 5, as well as subranges such as 2-5, 3-5, 2-3, 2-4, 1-4, etc.

The terms “formulation” and “composition” are used interchangeably herein.

It is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are described herein.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed. All patents and publications referred to herein are expressly incorporated by reference.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Additional Embodiments

Additional embodiments of the present disclosure are described in the following aspects.

Aspect 1. A storage stable topical composition comprising:
a) 1% to 20% by weight azelaic acid; and
b) 1% to 20% by weight of a urea agent
   dissolved in a non-aqueous skin-compatible solvent comprising polyol, C₍₃₋₆₎ alkanediol, glycol ether, dimethyl ether, or a combination thereof.

Aspect 2. The composition of aspect 1, wherein the composition is substantially free of water.

Aspect 3. The composition of aspect 1, wherein the composition comprises less than 1% by weight of water.

Aspect 4. The composition of aspect 1, wherein the composition is anhydrous.

Aspect 5. The composition of any one of aspects 1-5, wherein the composition is substantially free of volatile alcohols.

Aspect 6. The composition of any one of aspects 1-5, wherein the composition is substantially free of monohydric alcohols.

Aspect 7. The method of aspect 6, wherein the monohydric alcohol is selected from one or more of: ethyl alcohol, methyl alcohol, isopropyl alcohol, butyl alcohol, amyl alcohol, and cetyl alcohol.

Aspect 8. The composition of any one of aspects 1-7, wherein the urea agent is urea.

Aspect 9. The composition of any one of aspects 1-8, wherein the urea agent is hydroxyethyl urea.

Aspect 10. The composition of any one of aspects 1-9, wherein the urea agent comprises a mixture of urea and hydroxyethyl urea.

Aspect 11. The composition of any one of aspects 1-10, wherein the solvent is 1,3 propanediol.

Aspect 12. The composition of any one of aspects 1-11, wherein the solvent is a mixture of 1,3-propanediol and 1,2-hexanediol.

Aspect 13. The composition of any one of aspects 1-12, wherein the composition is storage stable (e.g., demonstrates less than 10 mol % degradation of the urea agent and azelaic acid) for 6 weeks at 40° C.±2° C. in a sealed container.

Aspect 14. The composition any one of aspects 1-13, wherein the composition is storage stable (e.g., demonstrates less than 10 mol % degradation of the urea agent and azelaic acid) for 6 months at 25° C.±2° C. in a multi-use container.

Aspect 15. The composition any one of aspects 1-14, wherein the composition is storage stable (e.g., demonstrates less than 10 mol % degradation of the urea agent and azelaic acid) for 12 months at 25° C.±2° C. in a multi-use container.

Aspect 16. The composition of any one of aspects 1-15, wherein the composition comprises 30% to 95% by weight of the non-aqueous solvent.

Aspect 17. The composition of any one of aspects 1-16, wherein the non-aqueous solvent is selected from 1,3 propanediol, 1,2 propanediol, 1,3 butanediol, 1,5 pentanediol, 1,2 hexanediol, 1,6 hexanediol, glycerol, diglycerol, ethoxydiglycol, dimethyl isosorbide and a combination thereof.

Aspect 18. The composition of aspect 17, wherein the solvent is 1,3 propanediol.

Aspect 19. The composition of any one of aspects 1-18, wherein the composition exhibits an azelaic acid degradation rate that is less than the azelaic acid degradation rate in the absence of the urea agent.

Aspect 20. The composition of any one of aspects 1-19, wherein the composition comprises 5% to 20% by weight of azelaic acid.

Aspect 21. The composition of any one of aspects 1-20, wherein the composition comprises 10% to 20% by weight of azelaic acid.

Aspect 22. The composition of any one of aspects 1-21, wherein the composition comprises 10% to 15% by weight (e.g., 10% to 12% by weight) of the azelaic acid.

Aspect 23. The composition of aspect 20, wherein the composition comprises 5% to 12% by weight of the azelaic acid.

Aspect 24. The composition of aspect 20, wherein the composition comprises 5% to 10% by weight of the azelaic acid.

Aspect 25. The composition of any one of aspects 1-19, wherein the composition comprises about 5% by weight of azelaic acid.

Aspect 26. The composition of any one of aspects 1-20, wherein the composition comprises about 10% by weight of azelaic acid.

Aspect 27. The composition of any one of aspects 1-26, wherein the composition comprises 1% to 10% by weight of the urea agent.

Aspect 28. The composition of any one of aspects 1-27, wherein the composition comprises 3% to 10% by weight of the urea agent.

Aspect 29. The composition of any one of aspects 1-28, wherein the composition comprises 5% to 10% by weight of the urea agent.

Aspect 30. The composition of any one of aspects 1-29, wherein the percent by weight ratio of the azelaic acid to urea agent to is 2.0 to 2.5.

Aspect 31. The composition of any one of aspects 1-30, further comprising 10% or less by weight in total of one or more optional additional components.

Aspect 32. The composition of aspect 31, wherein the one or more optional additional components are selected from tocopherols, tocotrienols (e.g., alpha, beta, delta and gamma tocopherols or alpha, beta, delta and gamma tocotrienols), ferulic acid, ascorbic acid, azelaic acid, hydroxy acids (e.g., salicylic acid), panthenol, Pinus pinaster bark extract, hyaluronic acid complex, cholesterol ester, cholesterol, ceramide, linoleic acid, linolenic acid, madecassoside, madecassoside asiaticoside, acetyl zingerone, bakuchiol, bis-ethylhexyl hydroxydimethoxy benzylmalonate, zinc oxide, and titanium dioxide.

Aspect 33. The composition of aspect 32, wherein the one or more additional components comprise one or more antioxidants selected from Vitis vinifera (Grape) Seed Extract, Camellia sinensis Leaf Extract, Quercus robur Wood Extract, and Pinus pinaster Bark Extract.

Aspect 34. The composition of aspect 33, wherein the one or more additional components comprise Vitis vinifera (Grape) Seed Extract, Camellia sinensis Leaf Extract, Quercus robur Wood Extract, and Pinus pinaster Bark Extract.

Aspect 35. The composition of any one of aspects 31-34, wherein the one or more additional components comprise ascorbic acid.

Aspect 36. The composition of aspect 35, wherein the composition comprises 10% or less by weight of ascorbic acid.

Aspect 37. The composition of aspect 36, wherein the composition comprises 5% to 10% by weight of ascorbic acid.

Aspect 38. The composition of any one of aspects 31-37, wherein the optional additional component composition further comprises ferulic acid.

Aspect 39. The composition of aspect 38, wherein the composition comprises 2% or less by weight of ferulic acid.

Aspect 40. The composition of any one of aspects 31-39, wherein the one or more optional additional components comprises vitamin E.

Aspect 41. The composition of aspect 40, wherein which the vitamin E is selected from alpha, beta, delta and gamma tocopherols and alpha, beta, delta and gamma tocotrienols, and combinations thereof.

Aspect 42. The composition of aspect 40, where the composition comprises 2% or less by weight of vitamin E.

Aspect 43. The composition of any one of aspects 31-42, wherein the one or more optional additional components comprises bis-ethylhexyl hydroxydimethoxy benzylmalonate.

Aspect 44. The composition of aspect 43, where in which the composition comprises 2% or less by weight of bis-ethylhexyl hydroxydimethoxy benzylmalonate.

Aspect 45. The composition of any one of aspects 31-44, wherein the one or more optional additional components is retinol.

Aspect 46. The composition of aspect 45, wherein the composition comprises 1% or less by weight of retinol.

Aspect 47. The composition of any one of aspects 31-46, wherein which the one or more optional additional components comprises bakuchiol.

Aspect 48. The composition of aspect 47, wherein the composition comprises 1% or less of bakuchiol.

Aspect 49. The composition of any one of aspects 31-48, wherein the one or more optional additional components comprise C10-C30 cholesterol/lanosterol esters.

Aspect 50. The composition of aspect 49, wherein the composition comprises 5% or less of C10-C30 cholesterol/lanosterol esters.

Aspect 51. The composition of any one of aspects 31-50, wherein the one or more optional additional components comprise madecassoside.

Aspect 52. The composition of aspect 51, wherein the composition comprises 1% or less by weight of madecassoside.

Aspect 53. The composition of any one of aspects 31-52, wherein the one or more optional additional components comprise glycyrrhetinic acid.

Aspect 54. The composition of aspect 53, wherein the composition comprises 1% or less of glycyrrhetinic acid.

Aspect 55. The composition of any one of aspects 31-54, wherein the one or more optional additional components comprise Pinus pinaster bark extract.

Aspect 56. The composition of aspect 55, wherein the composition comprises 2% or less by weight of Pinus pinaster bark extract.

Aspect 57. The composition of any one of aspects 31-56, wherein the one or more optional additional components comprise a ceramide.

Aspect 58. The composition of aspect 57, wherein the ceramide is selected from ceramide EOP, ceramide AP, ceramide NG, ceramide NP, ceramide NS, ceramide EOS, ceramide S, ceramide AS, and combinations thereof.

Aspect 59. The composition of aspect 57 or 58, wherein the composition comprises 2% or less by weight of ceramide.

Aspect 60. The composition of any one of aspects 31-59, wherein the one or more optional additional components comprise cholesterol.

Aspect 61. The composition of aspect 60, wherein the composition comprises less than 2% by weight of cholesterol.

Aspect 62. The composition of any one of aspects 31-61, wherein the one or more optional additional components comprise a free fatty acid.

Aspect 63. The composition of aspect 62, wherein the free fatty acid is selected from linoleic acid, linolenic acid, stearic acid, palmitic acid, oleic acid, alpha-linoleic, oleic acid, and combinations thereof.

Aspect 64. The composition of aspect 62 or 63, wherein the composition comprises less than 1% free fatty acid.

Aspect 65. The composition of any one of aspects 30 to 64, wherein the composition comprises:
a) 10% to 15% by weight azelaic acid; and
b) 5% to 10% by weight of a urea agent
   dissolved in a solvent comprising one or more C₍₃₋₆₎ alkanediols.

Aspect 66. The composition of aspect 65, wherein the composition comprises 12% by weight azelaic acid.

Aspect 67. The composition of aspect 65, wherein the composition comprises 10% by weight azelaic acid.

Aspect 68. The composition of any one of aspects 65 to 67, wherein the composition comprises 5% by weight of urea.

Aspect 69. The composition of any one of aspects 65 to 68, wherein the solvent is composed of 1,3-propanediol.

Aspect 70. The composition of any one of aspects 65 to 68, wherein the solvent is composed of a mixture of 1,3-propanediol and 1,2-hexanediol (e.g., in a ratio of at least 10:1, such as at least 15:1 or at least 20:1).

Aspect 71. The composition of aspect 65, wherein the composition is of Table 1 or 2.

Aspect 72. The composition of any one of aspects 1-71, wherein the composition is comprised in an emulsion that further comprises an external phase comprising 10% or more by weight of a silicone agent.

Aspect 73. A ready-to-use topical preparation in a multi-use container which is pre-filled with a storage stable topical composition according to any of the aspects 1-72, wherein the multi-use container comprises means for dispensing a single dose of the storage stable topical composition.

Aspect 74. The preparation of aspect 73, wherein the storage stable topical composition demonstrates less than 10 mol % degradation of the urea after storage for 6 weeks at 40° C.±2° C. in the container.

Aspect 75. The preparation of aspect 73, wherein the storage stable topical composition demonstrates less than 10 mol % degradation of the azelaic acid after storage for 6 months at 25° C.±2° C. in the container.

Aspect 76. The preparation of any one of aspects 73-75, wherein the storage stable topical composition is sealed in the container.

Aspect 77. The preparation of any one of aspects 73-76, wherein the container is placed in packaging.

Aspect 78. A process for producing a storage stable topical composition, the process comprising:
a. combining:
   1% to 20% by weight of azelaic acid;
   1% to 20% by weight of urea agent selected from urea, hydroxyethyl urea, and combination thereof; and
   30% to 95% by weight of a non-aqueous skin-compatible solvent selected from polyol, C₍₂₋₆₎ alkanediol, glycol ether, dimethyl ether, and a combination thereof; and optionally one or more additional agents;
   thereby dissolving the urea agent and the azelaic acid in the non-aqueous solvent to produce a homogenous solution that is storage stable.

Aspect 79. The process of aspect 78, further comprising suspending in the homogenous solution an emulsifying solution comprising 10% or more by weight of a silicone compound to produce a storage stable emulsion composition.

Aspect 80. The process of aspect 79, wherein the silicone compound is selected from cyclic, linear and branched silicones, a silicone crosspolymer, and a combination thereof.

Aspect 81. The process of aspect 78, wherein the one or more additional agents comprise ascorbic acid.

Aspect 82. The process of aspect 78, wherein the homogenous solution comprises 20% or less by weight of ascorbic acid.

Aspect 83. The process of aspect 82, wherein the homogenous solution comprises about 5% to about 10% by weight of ascorbic acid.

Aspect 84. The process of any one of aspects 78-83, wherein the one or more additional agents comprise ferulic acid.

Aspect 85. The process of aspect 84, wherein the homogenous solution comprises 2% or less by weight of ferulic acid.

Aspect 86. The process of aspect 78, wherein the one or more additional agents comprise:
0.5% to 2% by weight ferulic acid; and
0.5% to 2% by weight Pinus pinaster bark extract.

Aspect 87. The process of aspect 79, wherein the emulsion composition further comprises a lipid component.

Aspect 88. The process of aspect 87, wherein the lipid component is selected from cholesterol, ceramides, free fatty acids, and combinations thereof.

Aspect 89. The process of aspect 79, wherein the emulsion composition prevents or reduces precipitation of urea out of the emulsion composition.

Aspect 90. The process of aspect 79, wherein droplets of the homogenous solution is contained within the external phase of the emulsifying solution.

Aspect 91. A product produced by the process according to any one of aspects 78-90.

The invention is further defined by reference to the following examples. These examples are representative, and should not be construed to limit the scope of the invention.

### EXAMPLES

### Example 1: Assessment of Formulation Components

A series of experiments were performed to assess and optimize the components of the subject formulations.

### Solvents

1,3 propanediol, 1,2 propanediol, butylene glycol, pentylene glycol, and 1,2 hexanediol were identified as preferred solvents. 1,3 propanediol (trade name: Zemea) is inherently different from and preferable to the various polyols described. Below is a review of various polyols and reasons why 1,3 propanediol is unique and preferable:

1,3-propanediol, sometimes referred to in the art as propanediol, is unique in that it possesses a combination of gentleness on skin (even applied neat, or at 100% concentration), relatively low viscosity (and therefore perceived “lightness” on skin), environmental friendliness (not petroleum-derived), natural derivation (corn or sugar cane), low odor, and moderate ability to solubilize azelaic acid.

1,2-propanediol, otherwise referred to in the art as propylene glycol, although of low viscosity and possessing a moderate ability to solubilize azelaic acid, is well-known for inducing skin irritation and sensitivity. Additionally, it is derived from petroleum and possesses an unpleasant odor, reminiscent of acetone.

1,3-butanediol, otherwise referred to in the art as butylene glycol, is of low viscosity, possesses a moderate ability to solubilize azelaic acid, and is relatively gentle on skin. However, like propylene glycol, it is derived from petroleum (not environmentally friendly) and possesses an unpleasant odor, reminiscent of acetone.
also applicable to dipropylene glycol

1,5-pentanediol, otherwise referred to in the art as pentylene glycol, possesses a moderate ability to solubilize azelaic acid, low odor, and certain versions are not derived from petroleum but from sugarcane or corn. However, upon application to skin, it imparts a “heavier”, less desirable texture on skin. Additionally, its recommended use level is capped at 5%, limiting usage as a primary solvent.

1,2-hexanediol when used alone possesses a moderate ability to solubilize azelaic acid. However, the present compositions can include a 1,3 propanediol in combination with 1,2-hexanediol to effectively solubilize azelaic acid.

Glycerin and diglycerin, possess a moderate ability to solubilize azelaic acid, are relatively gentle on skin, are low-odor, and are not derived from petroleum. However, they are of a very viscous nature, and impart not only an undesirable, “heavy” texture on skin, but one that is exceedingly sticky.

Dimethyl isosorbide is relatively gentle on skin and not derived from petroleum, and imparts a “light”, not undesirable texture when applied to skin. However, it has a very limited ability to solubilize azelaic acid and possesses a slight, but noticeable chemical odor reminiscent of chlorine.

### Urea Agents

Urea is preferable to hydroxyethyl urea. There are a number of reasons for this:

Urea, when used in sufficient low concentrations (10-15% and below) in leave-on applications, possesses desirable humectant, barrier-repairing and very mild keratolytic properties, which in combination are very effective at improving the feel and look of dry and/or rough skin.

Urea is naturally present not only in the human body but specifically in the skin, where it acts as a natural moisturizing factor (NMF).

Hydroxyethyl urea possesses similar humectant properties, but not the same level of barrier-repairing and mild keratolytic properties of urea.

Additionally, hydroxyethyl urea may contain trace amounts of diethanolamine, which is listed as a potential carcinogen by California's Proposition 65, and requires a warning on products sold to consumers. For this reason, at least one manufacturer of hydroxyethyl urea has stated that it will discontinue production of this ingredient (AkzoNobel).

### Optional Additional Components

Additional ingredients were chosen for their compatibility with (e.g., miscibility in) 1,3 propanediol, 1,2 propanediol, and 1,3 butanediol. Additional notes and observations on each optional additional component are shown below.

### Panthenol (Pro-Vitamin B5)

This is a humectant that shows soothing and moisturizing properties for skin. Both enantiomers, D-panthenol and L-panthenol, are potent humectants. However, only D-panthenol is converted into pantothenic acid in the skin, which confers additional benefits to skin (wound healing, for example).

Research shows that it can reduce irritation to skin by other ingredients

Research also shows barrier-repairing ability (stimulation of physiologic lipid synthesis)

DL-panthenol is a racemic mixture of the two enantiomers; it is in powdered/crystal form.

D-panthenol is a viscous liquid.

DL-panthenol is freely soluble in 1,3 propanediol, 1,2 propanediol and 1,3 propanediol (up to 50%)

D-panthenol is also freely soluble in 1,3 propanediol, 1,2 propanediol and 1,3 propanediol, with no risk of recrystallization at any concentration (as it is already liquid at room temperature).

Inhibition of transepidermal water loss is apparent at concentrations of 1% and above.

### Hyaluronic Acid

Hyaluronic acid is a humectant that shows the ability to form a viscoelastic film on skin that prevents transepidermal water loss.

It is usually incorporated in aqueous solutions in its salt form, sodium hyaluronate

However, there is a raw material blend that is largely free from water, in which it is incorporated in a vehicle of glyceryl polymethacrylate, butylene glycol (1,3 butanediol), and natto gum (trade name Hydrafilm 3MW by The Innovation Company). This makes it compatible with the nonaqueous formulations of the present disclosure.

Documents from The Innovation Company show usage of this material up to 9.1% by weight of the final formula.

The chemical composition is as follows:
75-85% glyceryl polymethacrylate
15-20% butylene glycol
0.5-2% natto gum
0.5-2% hyaluronic acid
   Pinus pinaster Bark Extract

Research shows this ingredient's general antioxidant, anti-inflammatory and anti-acne properties.

Pycnogenol may be used as an alternative when Pinus pinaster bark extract is desired,
a material blend from Kinetik called Pantrofina Skin360 (PS360) is utilized in the subject formulations
PS360, unlike pycnogenol, is already in liquid form as it uses diglycerin as a solvent, making it very easy to incorporate

Additionally, Res Pharma Industriale provides in-vitro and clinical data to show effectiveness against free radical damage, inflammation and acne at a concentration of 0.5% by weight of PS360

The chemical composition is as follows:
90-95% diglycerin
5-10% Pinus pinaster bark extract

### Madecassoside/Asiaticoside

Centella asiatica extract is often used for its soothing properties.

Madecassoside and asiaticoside are a highly purified glycosylated triterpenes of Centella asiatica. It is sold by raw material supplier SEPPIC and Indena SpA, who share in-vitro and clinical data showing its anti-inflammatory and other effects on skin.

Clinical data from SEPPIC shows desirable ability to reduce erythema (skin redness) in concentrations of 0.2%.

At a concentration of 0.2%, madecassoside is soluble in 1,3 propanediol, 1,2 propanediol and 1,3 butanediol.

### Ferulic Acid

Ferulic acid is an antioxidant that shows a photoprotective effect on skin.

Ferulic acid is readily soluble in 1,3 propanediol, 1,2 propanediol, 1,3 butanediol and dimethyl isosorbide.

Isosorbide can increase the effectiveness of ferulic acid by enhancing skin penetration.

### Acetyl Zingerone

Acetyl zingerone is a broad-spectrum antioxidant that can prevent lipid peroxidation. It was engineered to be a more stable, more potent derivative of zingerone.

Sytheon provides in-vitro and clinical data showing its antioxidant, photoprotective, and anti-aging properties.

Acetyl zingerone may be used as a replacement for tocopherol.

Acetyl zingerone is readily soluble in 1,3 propanediol, 1,2 propanediol and 1,3 butanediol at the desired concentrations (0.5-1%), eliminating the need for emulsifiers as would be required for tocopherol.

### Glycyrrhizic Acid

Glycyrrhizic acid, like many other derivatives from licorice root (Glycyrrhiza Glabra, Glycyrrhiza Uralensis), shows anti-inflammatory, antioxidant and skin lightening properties.

Unlike 18β-glycyrrhetinic acid, glycyrrhizic acid shows solubility in 1,3-propanediol.

Other derivatives of licorice root can be used, such as dipotassium glycyrrhizate, monoammonium glycyrrhizate, etc.

### Example 2: Exemplary Formulations

### Assessment of Components

Exemplary non aqueous liquid compositions of this disclosure have been demonstrated to be stable against urea degradation, and stable against precipitation of components. The inventor discovered that compositions that include a combination of azelaic acid and urea provide for such stability and in addition have desirable physical properties when topically applied to skin (e.g., as described herein).

In order to determine a desirable ratio of azelaic acid to urea for certain compositions of this disclosure, the maximum concentration for azelaic acid that can be solubilized is first determined, with heat exposure, in a given solvent without precipitation upon cooling. Experiments revealed this concentration to be approximately 10 to 12% for 1,3 propanediol, propylene glycol (1,2 propanediol) and butylene glycol (1,3 butanediol), and significantly lower for dimethyl isosorbide.

Compositions having an azelaic acid concentration as low as 1 to 5% by weight can be prepared in cases where the polyol solvents used provide very low solubility, such as dimethyl isosorbide (DMI). Therefore, a mixture of propanediol and DMI, for example, can yield a percent by weight value of 10 to 12% by weight (e.g., maximum solubility of AzA), depending on the ratio of propanediol and DMI.

The ratio of azelaic acid to urea in the liquid composition can be 2.0 to 2.5 (e.g., 2.0), when the weight percent of azelaic acid is 10% to 12% dissolved in a solvent component that is composed of one or more C₍₂₋₆₎ alkanediols, such as 1,3-propanediol, or a mixture of 1,3-propanediol and 1,2-hexanediol. See e.g., the compositions of Tables 1 and 2.

In general, 1,3 propanediol is preferred over 1,2 propanediol, butylene glycol, pentylene glycol, or hexanediol. 1,3 propanediol is preferable to various polyols described in the art. Below is a review of various polyols and reasons why 1,3 propanediol is unique and preferable:

1,3 propanediol, otherwise referred to in the art as propanediol, is unique in that it possesses a combination of gentleness on skin (even applied neat, or at 100% concentration), relatively low viscosity (and therefore perceived “lightness” on skin), environmental friendliness (not petroleum-derived), natural derivation (corn or sugar cane), low odor, and moderate ability to solubilize azelaic acid.

1,2 propanediol, otherwise referred to in the art as propylene glycol, although of low viscosity and possessing a moderate ability to solubilize azelaic acid, induces skin irritation and sensitivity. Additionally, it is derived from petroleum and possesses an unpleasant odor, reminiscent of acetone.

1,3 butanediol, otherwise referred to in the art as butylene glycol, is of low viscosity, possesses a moderate ability to solubilize azelaic acid, and is relatively gentle on skin. However, like propylene glycol, it is derived from petroleum (not environmentally friendly) and possesses an unpleasant odor, reminiscent of acetone.

Note that these properties also apply to dipropylene glycol.

1,5 pentanediol, otherwise referred to in the art as pentylene glycol, possesses a moderate ability to solubilize azelaic acid, low odor, and certain versions are not derived from petroleum but from sugarcane or corn. However, upon application to skin, it imparts a “heavier”, less desirable texture on skin. Additionally, its recommended use level is generally capped at 5%, limiting usage as a primary solvent.

1,2 hexanediol possesses a moderate ability to solubilize azelaic acid. However, upon application to skin, it imparts a “heavier”, less desirable texture on skin, possesses an unpleasant odor reminiscent of acetone, and is derived from petroleum. Additionally, its recommended use level is capped at 10%, limiting usage as a primary solvent.

Glycerin and diglycerin, possess a low ability to solubilize azelaic acid, are relatively gentle on skin, are low-odor, and are not derived from petroleum. They are highly viscous, and impart not only an undesirable “heavy” texture on skin, but one that is exceedingly sticky.

Dimethyl isosorbide is relatively gentle on skin and not derived from petroleum, and imparts a “light”, not undesirable texture when applied to skin. However, it has a very limited ability to solubilize azelaic acid and possesses a slight, but noticeable chemical odor reminiscent of chlorine.

Urea is preferable to hydroxyethyl urea. There are a number of reasons for this, as summarized below:

Urea, when used in sufficient low concentrations (10-15% and below) in leave-on applications, possesses desirable humectant, barrier-repairing and very mild keratolytic properties, which in combination are very effective at improving the feel and look of dry and/or rough skin. Urea is naturally present not only in the human body but specifically in the skin, where it acts as a natural moisturizing factor (NMF).

Hydroxyethyl urea possesses similar humectant properties, but not the barrier-repairing and mild keratolytic properties of urea. Additionally, hydroxyethyl urea may contain trace amounts of diethanolamine, a potential carcinogen.

Additional ingredients can be included which are compatible with the azelaic acid/solvent/urea combination of interest.

The exemplary formulations of Table 1 were prepared and assessed as having desirable properties.

TABLE 1
Components of Exemplary Compositions (% by weight)

| Formulation | 1 | 2 | 3 | 4 | 5 | 6 |
|---|---|---|---|---|---|---|
| azelaic acid | 10% | 12% | 10% | 12% | 12% | 15% |
| urea/ | 5% | 5% | 5% | 5% | 5% | 5% |
| hydroxyethyl | | | | | | |
| urea | | | | | | |
| C3-C6 | 84.4% 1,3- | 82.4% 1,3- | 83% 1,- | 82.4% 1,3 | 79.4% 1,3 | 50% 1,3 |
| polyol | propanediol | propanediol | propanediol | propanediol | propanediol | propanedio |
| | | (and) 1,2- | (and) 1,2- | | and 3% 1,2 | 30% 1,2 |
| | | hexanediol | hexanediol | | Hexanediol | hexanediol |
| Additive 1 | .1% Vitis | .1% Vitis | .5% | 0.6% | 0.6% | |
| | Vinifera | Vinifera | Salicylic | Antioxidant | Antioxidant | |
| | (Grape) | (Grape) | Acid | blend (Vitis | blend (Vitis | |
| | Seed Extract | Seed Extract | | Vinifera | Vinifera | |
| | (and) | (and) | | (Grape) | (Grape) | |
| | Camellia | Camellia | | Seed Extract | Seed Extract | |
| | Sinensis | Sinensis | | (and) | (and) | |
| | Leaf Extract | Leaf Extract | | Camellia | Camellia | |
| | (and) | (and) | | Sinensis | Sinensis | |
| | Quercus | Quercus | | Leaf Extract | Leaf Extract | |
| | Robur | Robur | | (and) | (and) | |
| | Wood | Wood | | Quercus | Quercus | |
| | Extract | Extract | | Robur Wood | Robur Wood | |
| | | | | Extract, | Extract, | |
| | | | | Pinus | Pinus | |
| | | | | Pinaster | Pinaster | |
| | | | | Bark | Bark | |
| | | | | Extract) | Extract) | |
| Additive 2 | .5% Pinus | .5% Pinus | .5% Pinus | | | |
| | Pinaster | Pinaster | Pinaster | | | |
| | Bark | Bark | Bark | | | |
| | Extract | Extract | Extract | | | |
| Additive 3 | | | 1% Acetyl | | | |
| | | | Zingerone | | | |

Other variations: dimethyl isosorbide, caprylyl glycol or decylene glycol can be utilized as an alternative or additional solvent in the compositions of Table 1.

TABLE 2
Example Formulation

| Component | Percent by weight (%) |
|---|---|
| Propanediol | 80 |
| 1,2-Hexanediol | 3 |
| Urea | 5 |
| Salicylic Acid | 0.5% |
| Azelaic Acid | 10 |
| Acetyl Zingerone | 1 |
| Diglycerin (and) Pinus | 0.5 |
| Pinaster | |
| Bark Extract | |

### Example 3: Storage Stability

### Stability Method

The exemplary compositions are assessed for stability, e.g., chemical stability of a component (e.g., urea or azelaic acid) and/or physical stability (e.g., lack of precipitation or crystallization from a liquid composition). For example, the compositions are stored in containers over time, and are sampled at various time points, and assessed for levels of degradation of urea. Any breakdown of urea into ammonia can be assessed by a variety of methods. Urea degradation can be assessed by changes in pH over time, and/or by detection of ammonia via smell or another qualitative test.

The exemplary compositions are assessed for stability by comparison to a control composition that includes ethanol or isopropyl alcohol. The exemplary compositions are assessed for stability by comparison to a control composition that includes water at more than 2% by weight.

What is claimed is:

1. A storage stable topical composition comprising:
a. 1% to 20% by weight azelaic acid; and
b. 1% to 20% by weight of a urea agent
dissolved in a non-aqueous skin-compatible solvent comprising polyol, C₍₃₋₆₎ alkanediol, glycol ether, dimethyl ether, or a combination thereof.

2. The composition of claim 1, wherein the composition is substantially free of water.

3. The composition of claim 1, wherein the composition comprises less than 1% by weight of water.

4. The composition of claim 1, wherein the composition is anhydrous.

5. The composition of any one of claims 1-5, wherein the composition is substantially free of volatile alcohols.

6. The composition of any one of claims 1-5, wherein the composition is substantially free of monohydric alcohols.

7. The method of claim 6, wherein the monohydric alcohol is selected from one or more of: ethyl alcohol, methyl alcohol, isopropyl alcohol, butyl alcohol, amyl alcohol, and cetyl alcohol.

8. The composition of any one of claims 1-7, wherein the urea agent is urea.

9. The composition of any one of claims 1-8, wherein the urea agent is hydroxyethyl urea.

10. The composition of any one of claims 1-9, wherein the urea agent comprises a mixture of urea and hydroxyethyl urea.

11. The composition of any one of claims 1-10, wherein the solvent is 1,3 propanediol.

12. The composition of any one of claims 1-11, wherein the solvent is a mixture of 1,3-propanediol and 1,2-hexanediol.

13. The composition of any one of claims 1-12, wherein the composition is storage stable (e.g., demonstrates less than 10 mol % degradation of the urea agent and azelaic acid) for 6 weeks at 40° C.±2° C. in a sealed container.

14. The composition any one of claims 1-13, wherein the composition is storage stable (e.g., demonstrates less than 10 mol % degradation of the urea agent and azelaic acid) for 6 months at 25° C.±2° C. in a multi-use container.

15. The composition any one of claims 1-14, wherein the composition is storage stable (e.g., demonstrates less than 10 mol % degradation of the urea agent and azelaic acid) for 12 months at 25° C.±2° C. in a multi-use container.

16. The composition of any one of claims 1-15, wherein the composition comprises 30% to 95% by weight of the non-aqueous solvent.

17. The composition of any one of claims 1-16, wherein the non-aqueous solvent is selected from 1,3 propanediol, 1,2 propanediol, 1,3 butanediol, 1,5 pentanediol, 1,2 hexanediol, 1,6 hexanediol, glycerol, diglycerol, ethoxydiglycol, dimethyl isosorbide and a combination thereof.

18. The composition of claim 17, wherein the solvent is 1,3 propanediol.

19. The composition of any one of claims 1-18, wherein the composition exhibits an azelaic acid degradation rate that is less than the azelaic acid degradation rate in the absence of the urea agent.

20. The composition of any one of claims 1-19, wherein the composition comprises 5% to 20% by weight of azelaic acid.

21. The composition of any one of claims 1-20, wherein the composition comprises 10% to 20% by weight of azelaic acid.

22. The composition of any one of claims 1-21, wherein the composition comprises 10% to 15% by weight (e.g., 10% to 12% by weight) of the azelaic acid.

23. The composition of claim 20, wherein the composition comprises 5% to 12% by weight of the azelaic acid.

24. The composition of claim 20, wherein the composition comprises 5% to 10% by weight of the azelaic acid.

25. The composition of any one of claims 1-19, wherein the composition comprises about 5% by weight of azelaic acid.

26. The composition of any one of claims 1-20, wherein the composition comprises about 10% by weight of azelaic acid.

27. The composition of any one of claims 1-26, wherein the composition comprises 1% to 10% by weight of the urea agent.

28. The composition of any one of claims 1-27, wherein the composition comprises 3% to 10% by weight of the urea agent.

29. The composition of any one of claims 1-28, wherein the composition comprises 5% to 10% by weight of the urea agent.

30. The composition of any one of claims 1-29, wherein the percent by weight ratio of the azelaic acid to urea agent is 2.0 to 2.5.

31. The composition of any one of claims 1-30, further comprising 10% or less by weight in total of one or more optional additional components.

32. The composition of claim 31, wherein the one or more optional additional components are selected from tocopherols, tocotrienols (e.g., alpha, beta, delta and gamma tocopherols or alpha, beta, delta and gamma tocotrienols), ferulic acid, ascorbic acid, azelaic acid, hydroxy acids (e.g., salicylic acid), panthenol, Pinus pinaster bark extract, hyaluronic acid complex, cholesterol ester, cholesterol, ceramide, linoleic acid, linolenic acid, madecassoside, acetyl zingerone, bakuchiol, bis-ethylhexyl hydroxydimethoxy benzylmalonate, zinc oxide, and titanium dioxide.

33. The composition of claim 32, wherein the one or more additional components comprise one or more antioxidants selected from Vitis vinifera (Grape) Seed Extract, Camellia sinensis Leaf Extract, Quercus robur Wood Extract, and Pinus pinaster Bark Extract.

34. The composition of claim 33, wherein the one or more additional components comprise Vitis vinifera (Grape) Seed Extract, Camellia sinensis Leaf Extract, Quercus robur Wood Extract, and Pinus pinaster Bark Extract.

35. The composition of any one of claims 31-34, wherein the one or more additional components comprise ascorbic acid.

36. The composition of claim 35, wherein the composition comprises 10% or less by weight of ascorbic acid.

37. The composition of claim 36, wherein the composition comprises 5% to 10% by weight of ascorbic acid.

38. The composition of any one of claims 31-37, wherein the optional additional component composition further comprises ferulic acid.

39. The composition of claim 38, wherein the composition comprises 2% or less by weight of ferulic acid.

40. The composition of any one of claims 31-39, wherein the one or more optional additional components comprises vitamin E.

41. The composition of claim 40, wherein which the vitamin E is selected from alpha, beta, delta and gamma tocopherols and alpha, beta, delta and gamma tocotrienols, and combinations thereof.

42. The composition of claim 40, where the composition comprises 2% or less by weight of vitamin E.

43. The composition of any one of claims 31-42, wherein the one or more optional additional components comprises bis-ethylhexyl hydroxydimethoxy benzylmalonate.

44. The composition of claim 43, where in which the composition comprises 2% or less by weight of bis-ethylhexyl hydroxydimethoxy benzylmalonate.

45. The composition of any one of claims 31-44, wherein the one or more optional additional components is retinol.

46. The composition of claim 45, wherein the composition comprises 1% or less by weight of retinol.

47. The composition of any one of claims 31-46, wherein which the one or more optional additional components comprises bakuchiol.

48. The composition of claim 47, wherein the composition comprises 1% or less of bakuchiol.

49. The composition of any one of claims 31-48, wherein the one or more optional additional components comprise C10-C30 cholesterol/lanosterol esters.

50. The composition of claim 49, wherein the composition comprises 5% or less of C10-C30 cholesterol/lanosterol esters.

51. The composition of any one of claims 31-50, wherein the one or more optional additional components comprise madecassoside.

52. The composition of claim 51, wherein the composition comprises 1% or less by weight of madecassoside.

53. The composition of any one of claims 31-52, wherein the one or more optional additional components comprise glycyrrhetinic acid.

54. The composition of claim 53, wherein the composition comprises 1% or less of glycyrrhetinic acid.

55. The composition of any one of claims 31-54, wherein the one or more optional additional components comprise Pinus pinaster bark extract.

56. The composition of claim 55, wherein the composition comprises 2% or less by weight of Pinus pinaster bark extract.

57. The composition of any one of claims 31-56, wherein the one or more optional additional components comprise a ceramide.

58. The composition of claim 57, wherein the ceramide is selected from ceramide EOP, ceramide AP, ceramide NG, ceramide NP, ceramide NS, ceramide EOS, ceramide S, ceramide AS, and combinations thereof.

59. The composition of claim 57 or 58, wherein the composition comprises 2% or less by weight of ceramide.

60. The composition of any one of claims 31-59, wherein the one or more optional additional components comprise cholesterol.

61. The composition of claim 60, wherein the composition comprises less than 2% by weight of cholesterol.

62. The composition of any one of claims 31-61, wherein the one or more optional additional components comprise a free fatty acid.

63. The composition of claim 62, wherein the free fatty acid is selected from linoleic acid, linolenic acid, stearic acid, palmitic acid, oleic acid, alpha-linoleic, oleic acid, and combinations thereof.

64. The composition of claim 62 or 63, wherein the composition comprises less than 1% free fatty acid.

65. The composition of any one of claims 30 to 64, wherein the composition comprises:
a. 10% to 15% by weight azelaic acid; and
b. 5% to 10% by weight of a urea agent
dissolved in a solvent comprising one or more C₍₃₋₆₎ alkanediols.

66. The composition of claim 65, wherein the composition comprises 12% by weight azelaic acid.

67. The composition of claim 65, wherein the composition comprises 10% by weight azelaic acid.

68. The composition of any one of claims 65 to 67, wherein the composition comprises 5% by weight of urea.

69. The composition of any one of claims 65 to 68, wherein the solvent is composed of 1,3-propanediol.

70. The composition of any one of claims 65 to 68, wherein the solvent is composed of a mixture of 1,3-propanediol and 1,2-hexanediol (e.g., in a ratio of at least 10:1, such as at least 15:1 or at least 20:1).

71. The composition of claim 65, wherein the composition is of Table 1 or 2.

72. The composition of any one of claims 1-71, wherein the composition is comprised in an emulsion that further comprises an external phase comprising 10% or more by weight of a silicone agent.

73. A ready-to-use topical preparation in a multi-use container which is pre-filled with a storage stable topical composition according to any of the claims 1-72, wherein the multi-use container comprises means for dispensing a single dose of the storage stable topical composition.

74. The preparation of claim 73, wherein the storage stable topical composition demonstrates less than 10 mol % degradation of the urea after storage for 6 weeks at 40° C.±2° C. in the container.

75. The preparation of claim 73, wherein the storage stable topical composition demonstrates less than 10 mol % degradation of the azelaic acid after storage for 6 months at 25° C. 2° C. in the container.

76. The preparation of any one of claims 73-75, wherein the storage stable topical composition is sealed in the container.

77. The preparation of any one of claims 73-76, wherein the container is placed in packaging.

78. A process for producing a storage stable topical composition, the process comprising:
a. combining:
1% to 20% by weight of azelaic acid;
1% to 20% by weight of urea agent selected from urea, hydroxyethyl urea, and combination thereof; and
30% to 95% by weight of a non-aqueous skin-compatible solvent selected from polyol, C₍₂₋₆₎ alkanediol, glycol ether, dimethyl ether, and a combination thereof; and optionally one or more additional agents;
thereby dissolving the urea agent and the azelaic acid in the non-aqueous solvent to produce a homogenous solution that is storage stable.

79. The process of claim 78, further comprising suspending in the homogenous solution an emulsifying solution comprising 10% or more by weight of a silicone compound to produce a storage stable emulsion composition.

80. The process of claim 79, wherein the silicone compound is selected from cyclic, linear and branched silicones, a silicone crosspolymer, and a combination thereof.

81. The process of claim 78, wherein the one or more additional agents comprise ascorbic acid.

82. The process of claim 78, wherein the homogenous solution comprises 20% or less by weight of ascorbic acid.

83. The process of claim 82, wherein the homogenous solution comprises about 5% to about 10% by weight of ascorbic acid.

84. The process of any one of claims 78-83, wherein the one or more additional agents comprise ferulic acid.

85. The process of claim 84, wherein the homogenous solution comprises 2% or less by weight of ferulic acid.

86. The process of claim 78, wherein the one or more additional agents comprise:
0.5% to 2% by weight ferulic acid; and
0.5% to 2% by weight Pinus pinaster bark extract.

87. The process of claim 79, wherein the emulsion composition further comprises a lipid component.

88. The process of claim 87, wherein the lipid component is selected from cholesterol, ceramides, free fatty acids, and combinations thereof.

89. The process of claim 79, wherein the emulsion composition prevents or reduces precipitation of urea out of the emulsion composition.

90. The process of claim 79, wherein droplets of the homogenous solution is contained within the external phase of the emulsifying solution.

91. A product produced by the process according to any one of claims 78-90.
